(19) United States
(12) Patent Application Publication
(10) Pub. No.: US 2014/0288136 A1
(43) Pub. Date: Sep. 25, 2014

(54) METHODS AND USES OF CYTOCHROME P450 INHIBITORS

(71) Applicant: ANGION BIOMEDICA CORP, Uniondale, NY (US)

(72) Inventors: Lambertus J.W.M Oehlen, Westbury, NY (US); Bijoy Panicker, Holbrook, NY (US)

(21) Appl. No.: 14/187,282

(22) Filed: Feb. 23, 2014

Related U.S. Application Data
(63) Continuation of application No. 13/384,759, filed on Apr. 9, 2012, now abandoned.
(60) Provisional application No. 61/273,629, filed on Aug. 5, 2009.

(51) Int. Cl. A61K 31/4196; A61K 31/4172; A61K 31/4164

(52) U.S. Cl. CPC A61K 31/4196; A61K 31/4164; A61K 31/4172
USPC 514/383; 514/399

(57) ABSTRACT

Methods are provided for treating or preventing chronic obstructive pulmonary diseases such as emphysema, and fibrotic diseases including heart, liver, kidney and vascular diseases, by administering to a subject a pharmaceutical composition comprising a compound that inhibits cytochrome P450RA or CYP26.

### BACKGROUND OF THE INVENTION

Numerous debilitating diseases remain refractory to effective therapies despite intensive research efforts to identify treatments or even means to arrest their further progression in order to maintain a level of organ function to sustain a reasonable quality of life.

Emphysema is a major cause of death and disability in the United States and currently afflicts approximately 2 million people. Each year, this chronic lung disease causes or contributes to 100,000 deaths and costs more than $2.5 billion in health care expenses. Since it was reported centuries ago, an enormous effort has been directed towards fighting this devastating disease. Emphysema is a major form of chronic obstructive pulmonary disease (COPD) and is characterized by destruction of the alveolar wall, permanent enlargement of the airspaces and loss of lung recoil capability. Cigarette smoking is by far the single most important etiological factor of emphysema. Clinically, α1-antitrypsin (α1-AT) deficiency directly relates to and predisposes to the disease. Since about 40 years ago, the imbalance between protease and anti-protease activities, and its association with pulmonary inflammation has been a prevailing hypothesis for explaining the pathogenesis of emphysema. It suggests that elastolytic proteinases derived primarily from inflammatory cells (e.g. neutrophils and macrophages) overplays/outplays their counterparts—antiproteinases, and cause proteolytic destruction of the alveolar wall. Further, proteolysis and inflammation interact with each other in a positive feed-back manner, causing further damage to the alveoli and ultimately result in emphysema. More recently, the importance of pulmonary vascular endothelial cells and apoptosis in the pathogenesis of emphysema were also proposed, which was supported by the so-called non-inflammation emphysema model induced by chronic blockade of vascular endothelial growth factor receptor-2 (VEGF-R2). To date, the treatments for emphysema are primarily focused on halting the progressive processes or palliating the symptoms of the diseases, such as by using antibiotics, steroids, bronchodilators and protease inhibitors, with little evidence that they either alter the natural history of the disease or reduce mortality. Cessation of smoking is the only effective way to alter the rate of progression of emphysema; however, for many patients, the disease still persists long after smoking is stopped. Since the disease is unstoppable by medical intervention at the time of diagnosis, surgery seems to be the only intervention for the disease. Lung volume reduction surgery (LVRS) improves exercise capacity and yields a survival advantage for patients with predominantly upper-lobe emphysema and low base-line exercise capacity; however, it also increases mortality and offers negligible functional gain for other patients with non-upper lobe emphysema and high base-line exercise capacity. Overall, there is no significant difference of risk ratio between LVRS and conventional medical treatment over the entire emphysema patient population, as analyzed by the National Emphysema Treatment Trial Research Group. In addition, a review of pathologic specimens from LRVS patients raised the concern about the potential risk of carcinoma. The possibility of lung transplant is also limited by the availability of lung donors, and potential risks of infection and rejection. In summary, current treatments for emphysema are very limited, and more effective treatments are urgently needed.

Liver fibrosis is the scarring response of the liver to chronic liver injury; when fibrosis progresses to cirrhosis, morbid complications can develop. In fact, end-stage liver fibrosis or cirrhosis is the seventh leading cause of death in the United States, and afflicts hundreds of millions of people worldwide; deaths from end-stage liver disease in the United States are expected to triple over the next 10-15 years, mainly due to the hepatitis C epidemic1. In addition to the hepatitis C virus, many other forms of chronic liver injury also lead to end-stage liver disease and cirrhosis, including other viruses such as hepatitis B and delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency).

Stroke and cerebrovascular disease are a leading cause of morbidity and mortality in the US: at least 600,000 Americans develop strokes each year, and about 160,000 of these are fatal. Research on the pathophysiological basis of stroke has produced new paradigms for prevention and treatment, but translation of these approaches into improved clinical outcomes has proved to be painfully slow. Preventive strategies focus primarily on reducing or controlling risk factors such as diabetes, hypertension, cardiovascular disease, and lifestyle; in patients with severe stenosis, carotid endarterectomy may be indicated. Cerebral angioplasty is used investigationally, but the high restenosis rates observed following coronary angioplasty suggest this approach may pose unacceptable risk for many patients. Therapeutic strategies focus primarily on acute treatment to reduce injury in the ischemic penumbra, the region of reversibly damaged tissue surrounding an infarct. Thrombolytic therapy has been shown to improve perfusion to the ischemic penumbra, but it must be administered within three hours of the onset of infarction. Several neuroprotective agents that block specific tissue responses to ischemia are promising, but none have yet been approved for clinical use. While these therapeutic approaches limit damage in the ischemic penumbra, they do not address the underlying problem of inadequate blood supply due to occluded arteries. An alternative strategy is to induce formation of collateral blood vessels in the ischemic region; this occurs naturally in chronic ischemic conditions, but stimulation of vascularization via therapeutic angiogenesis has potential therapeutic benefit.

In addition to those mentioned above, other serious diseases such as ischemic heart disease, chronic renal dysfunction idiopathic pulmonary fibrosis, demyelinating diseases, and numerous others await more successful outcomes of research to provide effective treatments. It is towards the treatment of various serious diseases that the present invention is directed.

### SUMMARY OF THE INVENTION

The present invention is directed generally to the treatment and prevention of serious diseases such as but not limited to chronic obstructive pulmonary disease (COPD), including emphysema, chronic bronchitis and chronic asthma; fibrotic liver diseases; hepatic ischemia/reperfusion injury; stroke; ischemic heart diseases; renal diseases; idiopathic pulmonary fibrosis; demyelinating diseases of the nervous system such as multiple sclerosis; transplant and organ preservation; diabetes; amyotrophic lateral sclerosis; muscular dystrophy; and ischemic limb disease including peripheral vascular disease. Compounds that inhibit the activity of cytochrome P450, and in particular cytochrome P450RA1, also called CYP26, have been found to be beneficial for such purposes. Pharmaceutical compositions of such compounds, their isomers and pharmaceutically acceptable salts have been found to be useful for such therapeutic purposes. Described below are non-limiting examples of inhibitors of cytochrome P450 useful for these purposes, but the selections described below are merely illustrative and not intending to be limiting to the use of CYP26 inhibition in general to benefit patients with the aforementioned diseases among others.

In one embodiment, compounds useful for the purposes described herein are represented by formula (I)

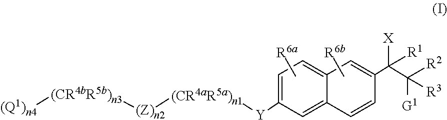

wherein G¹, R¹, R², R³, X, R^{6a}, R^{6b}, Y, R^{5a}, R^{4a}, Z, R^{5b}, R^{4b}, Q¹, n1, n2, n3 and n4 are as described generally and in classes and subclasses herein, tautomers thereof, Z and E isomers thereof, syn and anti isomers thereof, optically pure isomers thereof, pharmaceutically acceptable salts thereof, and pharmaceutical compositions thereof.

In another embodiment, compounds useful for the purposes described herein are represented by formula (I-A)

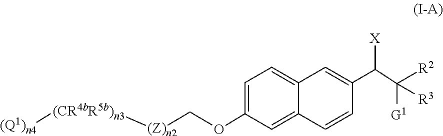

wherein G¹, R², R³, X, R^{4b}, R^{5b}, Z, Q¹, n2, n3 and n4 are as described generally and in classes and subclasses herein, tautomers thereof, Z and E isomers thereof, syn and anti isomers thereof, optically pure isomers thereof, pharmaceutically acceptable salts thereof, and pharmaceutical compositions thereof.

In another embodiment, compounds useful for the purposes described herein are represented by formula (I-B)

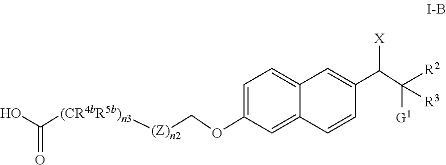

wherein G¹, R², R³, X, R^{4b}, R^{5b}, Z, n2 and n3 are as described generally and in classes and subclasses herein, tautomers thereof, Z and E isomers thereof, optically pure isomers thereof, and pharmaceutical compositions thereof.

In one embodiment, compounds useful for the purposes described herein are represented by formula (II)

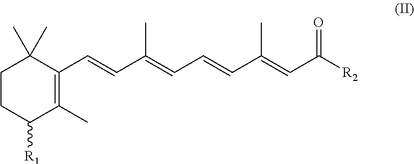

wherein R¹ and R² are as described generally and in classes and subclasses herein, tautomers thereof, Z and E isomers thereof, optically pure isomers thereof, and pharmaceutical compositions thereof.

Moreover, the compounds and compositions described herein are useful for the treatment, prevention, prophylaxis, ameliorization, stabilization or generally benefitting a disease or condition from among liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs, transplants or grafts; ischemia/reperfusion injury; stroke; cerebrovascular disease; myocardial ischemia; atherosclerosis; renal failure; renal fibrosis; idiopathic pulmonary fibrosis; wounds for acceleration of healing; vascularization of a damaged and/or ischemic organ, transplant or graft; amelioration of ischemia/reperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; renal failure secondary to chronic diabetes and/or hypertension; amytrophic lateral sclerosis, muscular dystrophy, acute liver failure, acute liver injury, scleroderma, diabetes mellitus, multiple sclerosis, trauma to the central nervous system, and hereditary neurodegenerative disorders including the leukodystrophies such as metachromatic leukodystrophy, Refsum's disease, adrenoleukodystrophy, Krabbe's disease, phenylketonuria, Canavan disease, Pelizaeus-Merzbacher disease and Alexander's disease.

### BRIEF DESCRIPTION OF THE FIGURES

FIG. 1 shows the effect of a compound of the invention on body weights in an elastase-induced mouse model of emphysema;

FIGS. 2A-B show the effect of a compound of the invention on pO₂ and oxygen saturation in an elastase-induced mouse model of emphysema;

FIGS. 3A-B show the effect of a compound of the invention on mean alveolar diameter in an elastase-induced mouse model of emphysema;

FIG. 4 shows the effect of a compound of the invention on body weights in the TAA-induced murine model of liver fibrosis;

FIG. 5A-B show the effect of a compound of the invention on the elevations of serum AST and ALT in the TAA-induced murine model of liver fibrosis;

FIG. 6 shows the effect of a compound of the invention on the elevations in αSMA expression in the TAA-induced murine model of liver fibrosis;

FIG. 7 shows the effect of a compound of the invention on the survival in the murine model of bleomycin-induced pulmonary fibrosis;

FIG. 8 shows the effect of a compound of the invention on the lung weights in the murine model of bleomycin-induced pulmonary fibrosis;

FIG. 9 shows the effect of a compound of the invention on the pulmonary hydroxyproline content in the murine model of bleomycin-induced pulmonary fibrosis;

FIG. 10 shows the effect of a compound of the invention on the αSMA expression in the murine model of bleomycin-induced pulmonary fibrosis;

FIG. 11A-B show the effect of a compound of the invention on the elevations of serum AST and ALT in the TAA-induced murine model of acute liver failure;

FIG. 12 shows the effect of a compound of the invention on the elevations in serum bilirubin in the TAA-induced murine model of acute liver failure; and

FIG. 13 shows the effect of a compound of the invention on elevations in liver cell apoptosis in the TAA-induced murine model of acute liver failure.

### DEFINITIONS

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, or alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms. “Lower alkenyl” and “lower alkynyl” respectively include corresponding 1-6 carbon moieties.

In certain embodiments, the alkyl, alkenyl and alkynyl groups employed in the invention contain 1-20; 2-20; 3-20; 4-20; 5-20; 6-20; 7-20 or 8-20 aliphatic carbon atoms. In certain other embodiments, the alkyl, alkenyl, and alkynyl groups employed in the invention contain 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl, alkenyl, and alkynyl groups employed in the invention contain 1-6; 2-6; 3-6; 4-6 or 5-6 aliphatic carbon atoms. In yet other embodiments, the alkyl, alkenyl, and alkynyl groups employed in the invention contain 1-4; 2-4 or 3-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alicyclic”, as used herein, refers to compounds that combine the properties of aliphatic and cyclic compounds and include but are not limited to monocyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The term “alkoxy” or “alkyloxy”, as used herein refers to a saturated (i.e., O-alkyl) or unsaturated (i.e., O-alkenyl and O-alkynyl) group attached to the parent molecular moiety through an oxygen atom. In certain embodiments, the alkyl group contains 1-20; 2-20; 3-20; 4-20; 5-20; 6-20; 7-20 or 8-20 aliphatic carbon atoms. In certain other embodiments, the alkyl group contains 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl group contains 1-6; 2-6; 3-6; 4-6 or 5-6 aliphatic carbon atoms. In yet other embodiments, the alkyl group contains 1-4; 2-4 or 3-4 aliphatic carbon atoms. Examples of alkoxy, include but are not limited to, methoxy, ethoxy, propoxy, isopropoxy, n-butoxy, i-butoxy, sec-butoxy, tert-butoxy, neopentoxy, n-hexoxy and the like.

The term “thioalkyl” as used herein refers to a saturated (i.e., S-alkyl) or unsaturated (i.e., S-alkenyl and S-alkynyl) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of thioalkyl include, but are not limited to, methylthio, ethylthio, propylthio, isopropylthio, n-butylthio, and the like.

The term “alkylamino” refers to a group having the structure —NHR′ wherein R′ is aliphatic or alicyclic, as defined herein. The term “aminoalkyl” refers to a group having the structure NH₂R′—, wherein R′ is aliphatic or alicyclic, as defined herein. In certain embodiments, the aliphatic or alicyclic group contains 1-20 aliphatic carbon atoms. In certain other embodiments, the aliphatic or alicyclic group contains 1-10 aliphatic carbon atoms. In still other embodiments, the aliphatic or alicyclic group contains 1-6 aliphatic carbon atoms. In yet other embodiments, the aliphatic or alicyclic group contains 1-4 aliphatic carbon atoms. In yet other embodiments, R′ is an alkyl, alkenyl, or alkynyl group containing 1-8 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aryl or heteroaryl substituents described above and herein may be substituted or unsubstituted. Additional examples of generally applicable substituents are illustrated by the specific embodiments shown in the Examples that are described herein.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

In general, the term “heteroaromatic moiety”, as used herein, refers to a stable mono- or polycyclic, unsaturated moiety having preferably 3-14 carbon atoms, each of which may be substituted or unsubstituted; and comprising at least one heteroatom selected from O, S and N within the ring (i.e., in place of a ring carbon atom). In certain embodiments, the term “heteroaromatic moiety” refers to a planar ring comprising at least one heteroatom, having p-orbitals perpendicular to the plane of the ring at each ring atom, and satisfying the Huckel rule where the number of pi electrons in the ring is (4n+2) wherein n is an integer.

It will also be appreciated that aromatic and heteroaromatic moieties, as defined herein may be attached via an alkyl or heteroalkyl moiety and thus also include (alkyl)aromatic, -heteroalkyl)aromatic, -(heteroalkyl)heteroaromatic, and (heteroalkyl)heteroaromatic moieties. Thus, as used herein, the phrases “aromatic or heteroaromatic moieties” and “aromatic, heteroaromatic, (alkyl)aromatic, -(heteroalkyl)aromatic, -(heteroalkyl)heteroaromatic, and -(heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl and the like.

The term “heteroaryl”, as used herein, does not differ significantly from the common meaning of the term in the art, and refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; hetero alkyl aryl; alkylhetero aryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom is a heteroatom selected from O, S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, pyrazolyl, imidazolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples or generally applicable substituents are illustrated by the specific embodiments shown in the Examples, which are described herein.

Additionally, it will be appreciated that any of the alicyclic or heterocyclic moieties described above and herein may comprise an aryl or heteroaryl moiety fused thereto. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}) or quaternary (—N⁺RₓR_{y}R_{z}) amine, where Rₓ, R_{y} and R_{z} are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “acyl”, as used herein, refers to a group having the general formula C(═O)R, where R is an aliphatic, alicyclic, heteroaliphatic, heterocyclic, aromatic or heteroaromatic moiety, as defined herein.

The term “C₂₋₆alkenylidene”, as used herein, refers to a substituted or unsubstituted, linear or branched unsaturated divalent radical consisting solely of carbon and hydrogen atoms, having from two to six carbon atoms, having a free valence “-” at both ends of the radical, and wherein the unsaturation is present only as double bonds and wherein a double bond can exist between the first carbon of the chain and the rest of the molecule.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the terms “cyclo alkyl”, “cycloalkenyl”, “cycloalkynyl”, “hetero cyclo alkyl”, “heterocyclealkenyl”, “heterocycloalkynyl”, “aromatic”, “heteroaromatic”, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

The term “tautomerization” refers to the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The term “tautomer” as used herein, refers to the compounds produced by the proton shift. For example, the compounds below can exist as a tautomer:

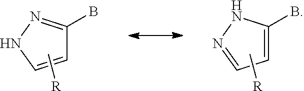

By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

Other definitions can be found in WO2005/007631, based on PCT/US2004/022282, and in WO2002/03912, based on PCT/01/16524, both contents of which are incorporated herein by reference in their entireties.

### DETAILED DESCRIPTION OF CERTAIN PREFERRED EMBODIMENTS OF THE INVENTION

The embodiments described herein are directed to prevention and treatment of numerous debilitating diseases that heretofore have remained refractory to effective therapies despite intensive research efforts to identify treatments or means to arrest their further progression in order to maintain a level of organ function to sustain a reasonable quality of life. Conditions and diseases such as but not limited to chronic obstructive pulmonary disease (COPD), including emphysema, chronic bronchitis and chronic asthma; fibrotic liver diseases; hepatic ischemia/reperfusion injury; stroke; ischemic heart diseases; renal diseases; idiopathic pulmonary fibrosis; demyelinating diseases of the nervous system such as multiple sclerosis; transplant and organ preservation; diabetes; amyotrophic lateral sclerosis; muscular dystrophy; and ischemic limb disease including peripheral vascular disease, are amenable to benefit by the compounds and compositions embodied herein. Such diseases and conditions are described in more detail below.

While Applicants have no duty to disclose the means by which the invention operates and are not bound thereto, compounds that inhibit the activity of cytochrome P450, and in particular cytochrome P450RA1, also called CYP26, have been found to be beneficial for such purposes. Pharmaceutical compositions of such compounds, their isomers and pharmaceutically acceptable salts, have been found to be useful for such therapeutic purposes. Described below are non-limiting examples of inhibitors of cytochrome P450 useful for these purposes, but the selections described below are merely illustrative and not intending to be limiting to the use of CYP26 inhibition in general to benefit patients with the aforementioned diseases.

Cytochrome P450 (CYP) is a family of enzymes is a very large and diverse superfamily of hemoproteins found in all domains of life. They use a plethora of both exogenous and endogenous compounds as substrates in enzymatic reactions. Usually they form part of multicomponent electron transfer chains, called P450-containing systems. Among the CYP family is CYP26 (also known as P450RA) that metabolizes retinoic acid. Based on the studies described herein, it has been found that inhibiting the activity of the CYP26 in vivo is an effective therapeutic approach to the treatment and prevention of a number of conditions and diseases as described herein by nonlimiting example. Any means of inhibiting CYP26 is embodied herein for the therapeutic purposes herein. Moreover, any condition, injury or disease modulated by the in-vivo level of all trans retenoic acid (ATRA) is encompassed herein. In certain embodiments, a compound or composition of the invention can be administered to inhibit CYP26 to increase endogenous ATRA, and an exogenous retinoic acid can be adminstered to further enhance the benefit of any of the conditions and diseases disclosed herein.

In one embodiment, the present invention is directed generally to the treatment and prevention of chronic obstructive pulmonary diseases. Chronic obstructive pulmonary disease (COPD) is estimated to affect 32 million persons in the United States and is the fourth leading cause of death in this country. Patients typically have symptoms of both chronic bronchitis and emphysema, but the classic triad also includes asthma. Most of the time COPD is secondary to tobacco abuse, although cystic fibrosis, alpha-1 antitrypsin deficiency, bronchiectasis, and some rare forms of bullous lung diseases may be causes as well. The invention is directed to all such causes of COPD.

Patients with COPD are susceptible to many insults that can lead rapidly to an acute deterioration superimposed on chronic disease. Quick and accurate recognition of these patients along with aggressive and prompt intervention may be the only action that prevents frank respiratory failure. COPD is a mixture of 3 separate disease processes that together form the complete clinical and pathophysiological picture. These processes are chronic bronchitis, emphysema and, to a lesser extent, asthma. Each case of COPD is unique in the blend of processes; however, 2 main types of the disease are recognized.

Chronic Bronchitis.

In this type, chronic bronchitis plays the major role. Chronic bronchitis is defined by excessive mucus production with airway obstruction and notable hyperplasia of mucus-producing glands. Damage to the endothelium impairs the mucociliary response that clears bacteria and mucus. Inflammation and secretions provide the obstructive component of chronic bronchitis. In contrast to emphysema, chronic bronchitis is associated with a relatively undamaged pulmonary capillary bed. Emphysema is present to a variable degree but usually is centrilobular rather than panlobular. The body responds by decreasing ventilation and increasing cardiac output. This V/Q mismatch results in rapid circulation in a poorly ventilated lung, leading to hypoxemia and polycythemia.

Eventually, hypercapnia and respiratory acidosis develop, leading to pulmonary artery vasoconstriction and cor pulmonale. With the ensuing hypoxemia, polycythemia, and increased CO₂ retention, these patients have signs of right heart failure and are known as “blue bloaters.”

Emphysema.

The second major type is that in which emphysema is the primary underlying process. Emphysema is defined by destruction of airways distal to the terminal bronchiole. Physiology of emphysema involves gradual destruction of alveolar septae and of the pulmonary capillary bed, leading to decreased ability to oxygenate blood. The body compensates with lowered cardiac output and hyperventilation. This V/Q mismatch results in relatively limited blood flow through a fairly well oxygenated lung with normal blood gases and pressures in the lung, in contrast to the situation in blue bloaters. Because of low cardiac output, however, the rest of the body suffers from tissue hypoxia and pulmonary cachexia. Eventually, these patients develop muscle wasting and weight loss and are identified as “pink puffers.”

In the US, two thirds of men and one fourth of women have emphysema at death. Approximately 8 million people have chronic bronchitis and 2 million have emphysema. COPD is the fourth leading cause of death in the United States, affecting 32 million adults. Men are more likely to have COPD than women, and COPD occurs predominantly in individuals older than 40 years.

History:

Patients with COPD present with a combination of signs and symptoms of chronic bronchitis, emphysema, and asthma. Symptoms include worsening dyspnea, progressive exercise intolerance, and alteration in mental status. In addition, some important clinical and historical differences can exist between the types of COPD. In the chronic bronchitis group, classic symptoms include the following: productive cough, with progression over time to intermittent dyspnea; frequent and recurrent pulmonary infections; and progressive cardiac/respiratory failure over time, with edema and weight gain. In the emphysema group, the history is somewhat different and may include the following set of classic symptoms: a long history of progressive dyspnea with late onset of nonproductive cough; occasional mucopurulent relapses; and eventual cachexia and respiratory failure.

Causes

In general, the vast majority of COPD cases are the direct result of tobacco abuse. While other causes are known, such as alpha-1 antitrypsin deficiency, cystic fibrosis, air pollution, occupational exposure (e.g., firefighters), and bronchiectasis, this is a disease process that is somewhat unique in its direct correlation to a human activity. The present invention is directed to benefiting COPD regardless of the cause or pathogenic mechanisms.

Thus, the present invention is directed generally to the treatment and prevention of chronic obstructive pulmonary disease as described above. COPD includes, by way of non-limiting example, emphysema, chronic bronchitis and chronic asthma. Such conditions may arise from, among other etiologies, cigarette smoking and other types of exposure to tobacco smoke including second-hand smoke.

In other embodiments of the invention, the compounds and compounds are useful for treating diseases in which abnormal or excessive fibrosis is a pathophysiologic component. Non-limiting examples of fibrotic diseases including diseases with a fibrotic component are described below.

In another aspect, the invention is directed to the treatment or prevention of fibrotic liver disease. Liver fibrosis is the scarring response of the liver to chronic liver injury; when fibrosis progresses to cirrhosis, morbid complications can develop. In fact, end-stage liver fibrosis or cirrhosis is the seventh leading cause of death in the United States, and afflicts hundreds of millions of people worldwide; deaths from end-stage liver disease in the United States are expected to triple over the next 10-15 years, mainly due to the hepatitis C epidemic. In addition to the hepatitis C virus, many other forms of chronic liver injury also lead to end-stage liver disease and cirrhosis, including other viruses such as hepatitis B and delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency).

Treatment of liver fibrosis has focused to date on eliminating the primary injury. For extrahepatic obstructions, biliary decompression is the recommended mode of treatment whereas patients with Wilson's disease are treated with zinc acetate. In chronic hepatitis C infection, interferon has been used as antiviral therapies with limited response: ˜20% when used alone or ˜50% response when used in combination with ribavirin. In addition to the low-level of response, treatment with interferon with or without ribavirin is associated with numerous severe side effects including neutropenia, thrombocytopenia, anemia, depression, generalized fatigue and flu-like symptoms, which are sufficiently significant to necessitate cessation of therapy. Treatments for other chronic liver diseases such as hepatitis B, autoimmune hepatitis and Wilson's disease are also associated with many side effects, while primary biliary cirrhosis, primary sclerosing cholangitis and non-alcoholic fatty liver disease have no effective treatment other than liver transplantation.

The advantage of treating fibrosis rather than only the underlying etiology, is that antifibrotic therapies should be broadly applicable across the full spectrum of chronic liver diseases. While transplantation is currently the most effective cure for liver fibrosis, mounting evidence indicates that not only fibrosis, but even cirrhosis is reversible. Unfortunately patients often present with advanced stages of fibrosis and cirrhosis, when many therapies such as antivirals can no longer be safely used due to their side effect profile. Such patients would benefit enormously from effective antifibrotic therapy, because attenuating or reversing fibrosis may prevent many late stage complications such as infection, asciites, and loss of liver function and preclude the need for liver transplantation. The compounds of the invention are beneficial for the treatment of the foregoing conditions, and generally are antifibrotic and/or antiapoptotic agents for this and other organ or tissues.

In another aspect, the invention is directed to the prevention or treatment of hepatic ischemia-reperfusion injury. Currently, transplantation is the most effective therapeutic strategy for liver fibrosis. However, in spite of the significant improvement in clinical outcome during the last decade, liver dysfunction or failure is still a significant clinical problem after transplantation surgery. Ischemia-reperfusion (IR) injury to the liver is a major alloantigen-independent component affecting transplantation outcome, causing up to 10% of early organ failure, and leading to the higher incidence of both acute and chronic rejection. Furthermore, given the dramatic organ shortage for transplantation, surgeons are forced to consider cadaveric or steatotic grafts or other marginal livers, which have a higher susceptibility to reperfusion injury. In addition to transplantation surgery, liver IR injury is manifested in clinical situations such as tissue resections (Pringle maneuver), and hemorrhagic shock.

The damage to the postischemic liver represents a continuum of processes that culminate in hepatocellular injury. Ischemia activates Kupffer cells, which are the main sources of vascular reactive oxygen species (ROS) formation during the initial reperfusion period. In addition to Kupffer cell-induced oxidant stress, with increasing length of the ischemic episode, intracellular generation of ROS by xanthine oxidase and in particular mitochondria may also contribute to liver dysfunction and cell injury during reperfusion. Endogenous antioxidant compounds, such as superoxide dismutase, catalase, glutathione, alphatocopherol, and beta-carotene, may all limit the effects of oxidant injury but these systems can quickly become overwhelmed by large quantities of ROS. Work by Lemasters and colleagues, has indicated that in addition to formation of ROS, intracellular calcium dyshomeostasis is a key contributor to liver IR injury. Cell death of hepatocytes and endothelial cells in this setting is characterized by swelling of cells and their organelles, release of cell contents, eosinophilia, karyolysis, and induction of inflammation, characteristic of oncotic necrosis. More recent reports indicate that liver cells also die by apoptosis, which is morphologically characterized by cell shrinkage, formation of apoptotic bodies with intact cell organelles and absence of an inflammatory response.

Indeed, minimizing the adverse effects of IR injury could significantly increase the number of patients that may successfully undergo liver transplantation. Pharmacologic interventions that reduce cell death and/or enhance organ regeneration represent a therapeutic approach to improve clinical outcome in liver transplantation, liver surgery with vascular exclusion and trauma and can therefore reduce recipient/patient morbidity and mortality. The compounds of the invention are beneficial for the treatment of the foregoing conditions.

In another aspect, the invention is directed to the prevention or treatment of cerebral infarction. Stroke and cerebrovascular disease are a leading cause of morbidity and mortality in the US: at least 600,000 Americans develop strokes each year, and about 160,000 of these are fatal. Research on the pathophysiological basis of stroke has produced new paradigms for prevention and treatment, but translation of these approaches into improved clinical outcomes has proved to be painfully slow. Preventive strategies focus primarily on reducing or controlling risk factors such as diabetes, hypertension, cardiovascular disease, and lifestyle; in patients with severe stenosis, carotid endarterectomy may be indicated. Cerebral angioplasty is used investigationally, but the high restenosis rates observed following coronary angioplasty suggest this approach may pose unacceptable risk for many patients. Therapeutic strategies focus primarily on acute treatment to reduce injury in the ischemic penumbra, the region of reversibly damaged tissue surrounding an infarct. Thrombolytic therapy has been shown to improve perfusion to the ischemic penumbra, but it must be administered within three hours of the onset of infarction. Several neuroprotective agents that block specific tissue responses to ischemia are promising, but none have yet been approved for clinical use. While these therapeutic approaches limit damage in the ischemic penumbra, they do not address the underlying problem of inadequate blood supply due to occluded arteries. An alternative strategy is to induce formation of collateral blood vessels in the ischemic region; this occurs naturally in chronic ischemic conditions, but stimulation of vascularization via therapeutic angiogenesis has potential therapeutic benefit.

Recent advances in imaging have confirmed the pathophysiological basis of the clinical observations of evolving stroke. Analysis of impaired cerebral blood flow (CBF) in the region of an arterial occlusion supports the hypothesis that a central region of very low CBF, the ischemic core, is irreversibly damaged, but damage in surrounding or intermixed zones where CBF is of less severely reduced, the ischemic penumbra, can be limited by timely reperfusion. Plate recently reviewed the evidence suggesting that therapeutic angiogenesis may be useful for treatment or prevention of stroke. First, analysis of cerebral vasculature in stroke patients showed a strong correlation between blood vessel density and survival and a higher density of microvessels in the ischemic hemisphere compared to the contralateral region. Second, studies in experimental models of cerebral ischemia indicate expression of angiogenic growth factors such as vascular endothelial growth factor (VEGF) or HGF/SF is induced rapidly in ischemic brain tissue. Third, administration of VEGF or HGF/SF can reduce neuronal damage and infarct volume in animal models. Similar evidence provided the rationale for developing therapeutic angiogenesis for treating peripheral and myocardial ischemia, which has been shown to produce clinical improvements in early studies in humans. The compounds of the invention, having similar antifibrotic properties, are beneficial for the treatment of the foregoing conditions.

In another aspect, the invention is directed to the prevention or treatment of ischemic heart disease, which is a leading cause of morbidity and mortality in the US, afflicting millions of Americans each year at a cost expected to exceed $300 billion/year. Numerous pharmacological and interventional approaches are being developed to improve treatment of ischemic heart disease including reduction of modifiable risk factors, improved revascularization procedures, and therapies to halt progression and/or induce regression of atherosclerosis. One of the most exciting areas of research for the treatment of myocardial ischemia is therapeutic angiogenesis. Recent studies support the concept that administration of angiogenic growth factors, either by gene transfer or as a recombinant protein, augments nutrient perfusion through neovascularization. The newly developed, supplemental collateral blood vessels constitute endogenous bypass conduits around occluded native arteries, improving perfusion to ischemic tissue. The compounds of the invention are beneficial for the treatment of the foregoing conditions.

In another aspect, the invention is directed to the prevention or treatment of renal diseases. Chronic renal dysfunction is a progressive, degenerative disorder that ultimately results in acute renal failure and requires dialysis as an intervention, and renal transplantation as the only potential cure. Initiating conditions of renal dysfunction include ischemia, diabetes, underlying cardiovascular disease, or renal toxicity associated with certain chemotherapeutics, antibiotics, and radiocontrast agents. Most end-stage pathological changes include extensive fibrinogenesis, epithelial atrophy, and inflammatory cell infiltration into the kidneys.

Acute renal failure is often a complication of diseases including diabetes or renal ischemia, procedures such as heminephrectomy, or as a side effect of therapeutics administered to treat disease. The widely prescribed anti-tumor drug cis-diamminedichloroplatinum (cisplatin), for example, has side effects that include a high incidence of nephrotoxicity and renal dysfunction, mainly in the form of renal tubular damage that leads to impaired glomerular filtration. Administration of gentamicin, an aminoglycoside antibiotic, or cyclosporin A, a potent immunosuppressive compound, causes similar nephrotoxicity. The serious side effects of these effective drugs restrict their use. The development of agents that protect renal function and enhance renal regeneration after administration of nephrotoxic drugs will be of substantial benefit to numerous patients, especially those with malignant tumors, and may allow the maximal therapeutic potentials of these drugs to be realized. The compounds of the invention are beneficial for the treatment of the renal diseases mentioned above.

In another aspect, the invention is directed to the prevention or treatment of lung (pulmonary) fibrosis. Idiopathic pulmonary fibrosis (IPF) accounts for a majority of chronic interstitial lung diseases, and has an estimated incidence rate of 10.7 cases for 100,000 per year, with an estimated mortality of 50-70%. IPF is characterized by an abnormal deposition of collagen in the lung with an unknown etiology. Although the precise sequence of the pathogenic sequelae is unknown, disease progression involves epithelial injury and activation, formation of distinctive subepithelial fibroblast/myofibroblast foci, and excessive extracellular matrix accumulation. The development of this pathological process is preceded by an inflammatory response, often dominated by macrophages and lymphocytes, which is mediated by the local release of chemoattractant factors and upregulation of cell-surface adhesion molecules. Lung injury leads to vasodilatation and leakage of plasma proteins into interstitial and alveolar spaces, as well as activation of the coagulation cascade and deposition of fibrin. Fibroblasts migrate into this provisional fibrin matrix where they synthesize extracellular matrix molecules. In non-pathogenic conditions, excess fibrin is usually degraded by plasmin, a proteinase that also has a role in the activation of matrix metalloproteinases (MMPs). Activated MMPs degrade extracellular matrix and participate in fibrin removal, resulting in the clearance of the alveolar spaces and the ultimate restoration of injured tissues. In pathological conditions, however, these processes can lead to progressive and irreversible changes in lung architecture, resulting in progressive respiratory insufficiency and an almost universally terminal outcome in a relatively short period of time. Fibrosis is the final common pathway of a variety of lung disorders, and in this context, the diagnosis of pulmonary fibrosis implies the recognition of an advanced stage in the evolution of a complex process of abnormal repair. While many studies have focused on inflammatory mechanisms for initiating the fibrotic response, the synthesis and degradation the extracellular matrix represent the central event of the disease. It is this process that presents a very attractive site of therapeutic intervention.

The course of IPF is characterized by progressive respiratory insufficiency, leading to death within 3 to 8 years from the onset of symptoms. Management of interstitial lung disease in general, and in particular idiopathic pulmonary fibrosis, is difficult, unpredictable and unsatisfactory. Attempts have been made to use antiinflammatory therapy to reverse inflammation, relief, stop disease progression and prolong survival. Corticosteroids are the most frequently used antiinflammatory agents and have been the mainstay of therapy for IPF for more than four decades, but the efficacy of this approach is unproven, and toxicities are substantial. No studies have compared differing dosages or duration of corticosteroid treatment in matched patients. Interpretation of therapy efficacy is obscured by several factors including heterogeneous patient populations, inclusion of patients with histologic entities other than usual interstitial pneumonia, lack of objective, validated endpoints, and different criteria for “response.” Cytotoxic drugs such as Azathioprine and cyclophosohamide have also being used in combination with low dose oral corticosteroids. The results of such treatments vary from no improvement to significant prolongation of survival. Overall, currently available treatments for lung fibrosis are sub-optimal. Potential new therapies have emerged from the use of animal models of pulmonary fibrosis and recent advances in the cellular and molecular biology of inflammatory reactions. Such therapies involve the use of cytokines, oxidants and growth factors that are elaborated during the fibrotic reaction. Despite the use of newer strategies for treatment, the overall prognosis for patients with interstitial lung disease has had little quantifiable change, and the population survival remains unchanged for the last 30 years. Interferon gamma (IFN) may be effective in the treatment of IPF in some patients but its role is controversial. Literature indicated that IFN-gamma may be involved in small airway disease in silicotic lung. Others showed that IFN gamma mediates, bleomycin-induced pulmonary inflammation and fibrosis. The compounds of the invention are beneficial for the treatment of the foregoing condition, among other fibrotic diseases.

In another aspect, the invention is directed to the prevention or treatment of demyelinating diseases, which are those diseases in which myelin is the primary target. They fall into two main groups: acquired diseases (i.e., multiple sclerosis) and hereditary neurodegenerative disorders (i.e., the leukodystrophies). Although their causes and etiologies are different, they have the same outcome: central nervous system (CNS) demyelination. Without myelin, nerve impulses are slowed or stopped, leading to a constellation of neurological symptoms. Multiple sclerosis (MS) is the most common demyelinating disease, which usually manifests itself between the 20th and 50th years of life. Current estimates are that approximately 2.5 million people worldwide have MS, with between 250,000 and 350,000 cases in the United States, 50,000 cases in Canada, 130,000 cases in Germany, 85,000 cases in the United Kingdom, 75,000 cases in France, 50,000 cases in Italy, and 11,000 cases in Switzerland.

MS attacks the white matter of the CNS. In its classic manifestation (90% of all cases), it is characterized by alternating relapsing/remitting phases with the periods of remission growing shorter over time. Its symptoms include any combination of spastic paraparesis, unsteady gait, diplopia, and incontinence.

Other demyelinating diseases include leukodystrophies: metachromatic leukodystrophy, Refsum's disease, adrenoleukodystrophy, Krabbe's disease, phenylketonuria, Canavan disease, Pelizaeus-Merzbacher disease and Alexander's disease. The first six are storage disorders. The lack or the malfunctioning of an enzyme causes a toxic buildup of chemical substances. In Pelizaeus-Merzbacher disease myelin is never formed (dysmyelination) because of a mutation in the gene that produces a basic protein of CNS myelin. The etiology of Alexander's disease remains largely unknown.

The foregoing are merely exemplary and non-limiting examples of diseases and conditions in which an inhibitor of CYP26 is effective in the prevention, treatment, amelioration, prophylaxis, stabilization, or otherwise benefitting an individual or patient suffering from or at risk for developing, such a condition of disease. The compounds and compositions described herein are useful for the treatment, prevention, prophylaxis, ameliorization, stabilization or generally benefitting a disease or condition from among liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs, transplants or grafts; ischemia/reperfusion injury; stroke; cerebrovascular disease; myocardial ischemia; atherosclerosis; renal failure; renal fibrosis; idiopathic pulmonary fibrosis; wounds for acceleration of healing; vascularization of a damaged and/or ischemic organ, transplant or graft; amelioration of ischemia/reperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; renal failure secondary to chronic diabetes and/or hypertension; amytrophic lateral sclerosis, muscular dystrophy, acute liver failure, acute liver injury, scleroderma, diabetes mellitus, multiple sclerosis, trauma to the central nervous system, and hereditary neurodegenerative disorders including the leukodystrophies such as metachromatic leukodystrophy, Refsum's disease, adrenoleukodystrophy, Krabbe's disease, phenylketonuria, Canavan disease, Pelizaeus-Merzbacher disease and Alexander's disease.

### Exemplary Assays

Efficacy of the compounds of the invention on the aforementioned disorders and diseases or the potential to be of benefit for the prophylaxis or treatment thereof may be demonstrated in various studies, ranging from biochemical effects evaluated in vitro and effects on cells in culture, to in-vivo models of disease, wherein direct clinical manifestations of the disease can be observed and measured, or wherein early structural and/or functional events occur that are established to be involved in the initiation or progression of the disease. The positive effects of the compounds of the invention have been demonstrated in a variety of such assays and models, for a number of diseases and disorders. One skilled in the art can readily determine following the guidance described herein whether a compound of the invention is an inhibitor of CYP mimic and is useful therapeutically.

Hepatic Disease. 1. Antifibrotic Activity in Hepatic Stellate Cells. Serum starved (activated) LX2 cells (an immortalized human hepatic stellate cell line) that are treated with a compound of the invention show a decrease in collagen I mRNA expression, as well as expression of other fibrotic marker genes, related to significant antifibrotic activity.

2. Liver Disease endpoints. The rat model of thioacetamide (TAA)-induced liver fibrosis and the rat bile duct ligation model of fibrosis shows improvements by the compounds of the invention, in a panel of functional and histological tests: gross morphology, mass, portal pressure, presence of ascites, enzymes (AST, ALT), collagen content, interstitial fibrosis and alpha-smooth muscle actin and MMP-2.

Protection Against Renal Dysfunction. 1. Clinical model: arterial occlusion. In a mouse model of transient unilateral renal artery occlusion, male ICR mice are anesthetized and the left renal artery occluded with a microvascular clamp. After 30 minutes, the clamp is removed and the kidney allowed to reperfuse. Ten minutes into reperfusion the nonischemic contralateral kidney is excised. Animals are treated daily with vehicle or compound of the invention (1 mg/kg, i.p.) until the day of sacrifice. Serum creatinine, BUN and urine protein levels, measured at 1, 4 and 7 days postischemia are used to determine the ability of compounds of the invention to restore function to injured kidneys. In order to create a more severe renal injury, animals are subjected to 45 minutes of ischemia.

2. Protection against HgCl₂-induced renal injury. In a study mice are injected with a high dose of HgCl₂ (7 mg/kg, s.c.) and divided into treatment groups. Animals in the first group receive vehicle or a compound of the invention (1 mg/kg, i.p.) on the day of toxin injection and daily thereafter for 3 days, and are euthanized on day 4. Blood samples that are collected prior to HgCl₂ injection, on day 2 and on day 4 are analyzed for serum creatinine. In the second group, treatment with vehicle or compound begins on the day following toxin injection (i.e., 24h delayed treatment) and daily thereafter until day 6. Mice are euthanized on day 7. Blood samples collected prior to HgCl₂ injection, on day 4 and day 7 are analyzed for serum creatinine and BUN. Serum creatinine, BUN, and envelopment of tubular necrosis are measured to indicate positive clinical activity.

3. Protection against ureteral obstruction. The effects of the compounds of invention on renal injury secondary to ureteral obstruction are examined in a mouse model of transient unilateral renal artery occlusion. Kidneys from mice subject to unilateral ureteral obstruction for 2 weeks are examined for histological evidence of injury and protection by compound treatment. Immunohistochemical staining is performed for fibronectin, proliferating cell nuclear antigen, and TUNEL (for an assessment of apoptosis). Trichrome staining is also performed to assess the extent of collagen formation as an indication of interstitial fibrosis.

Cerebral infarction/stroke. 1. Neuroprotective Effects in Brain Tissue. Cerebral infarction is induced in rats by middle cerebral artery occlusion (MCAO) for 24 hr. Test compound or vehicle is administered by i.p. at 2 mg/kg at −24, 0, and 8 hr. Sections of the brain are then examined for cell death by staining with a tetrazolium compound (2,3,5-Triphenyl-2H-tetrazolium chloride, or TTC). Normal rat brains exhibit a red staining due to TTC reduction whereas areas containing dead cells are white.

Myocardial Infarction. 1. Ability of the compounds of the invention to inhibit apoptosis in a rat model of myocardial infarction (as mentioned above). Hearts from rats subject to left coronary artery ligation are treated with compound (or vehicle control) by direct injection and 24 hours later sectioned and TUNEL stained. Treatment is associated with a significant reduction in the number of apoptotic nuclei.

2. Clinical model. In a rat ischemia model, myocardial infarction is induced by anterior descending artery occlusion. The infarction is evident by an increase in positive TUNEL staining, indicating DNA fragmentation in late-stage apoptosis. Treatment with compounds of the invention greatly reduces the extent of TUNEL staining.

Transplantation and Organ Preservation. 1. The viability of organs and tissues harvested and transported for transplant is currently optimally maintained by bathing and transport in storage solutions such as the University of Wisconsin (UW) cold storage solution (100 mM KH2PO₄, 5 mM MgSO₄ 100 mM potassium lactobionate, 1 mM allopurinol, 3 mM glutathione, 5 mM adenosine, 30 mM raffinose, 50 g/liter of hydroxyethyl starch, 40 units/liter of insulin, 16 mg/liter of dexamethasone, 200,000 units/liter of penicillin, pH 7.4; 320-330 mOsM) (Ploeg R J, Goossens D, Vreugdenhil P, McAnulty J F, Southard J H, Belzer F O. Successful 72-hour cold storage kidney preservation with UW solution. Transplant Proc. 1988 February; 20(1 Suppl 1):935-8.). To further enhance the viability of transplanted organs and tissues, inhibit apoptosis and promote vascularization thereof, one or more compounds of the invention may in included in this or any other storage solution, as well as perfused into the donor or donor organ prior to harvesting, and administered to the recipient systemically and/or locally into the transplanted organ or transplant site.

Lung fibrosis. 1. In order to assess the effects of test compounds on pulmonary fibrosis a well-established mouse model of bleomycin-induced lung injury is used. Male C57BL/6 mice (20-30 g, n=10/group) are treated with bleomycin (0.06 U/20 gram body weight) or saline via intratracheal administration. Bleomycin-treated mice are divided into 2 groups. Compounds of the invention (1 mg/kg, i.p.) or vehicle is administered daily until sacrifice on day 12. Right lung samples from the mice are then harvested for analysis. Tissues are sectioned and stained with modified Masson's Trichrome and are analyzed for interstitial fibrosis. The Ashcroft scale is used to obtain a numerical fibrotic score with each specimen being scored independently by two histopathologists, and the mean of their individual scores considered as the fibrotic score.

2. The porcine pancreatic elastase (PPE)-induced emphysema murine model can be used. For the induction of emphysema, the protocol described in the literature by Takahashi and colleagues (Takahashi S, Nakamura H, Seki M et al. Reversal of elastase-induced pulmonary emphysema and promotion of alveolar epithelial cell proliferation by simvastatin in mice. Am J Physiol Lung Cell Mol Physiol 2008 May; 294(5):L882-L890) is followed. Porcine pancreatic elastase (PPE) is obtained from Sigma (St. Louis, Mo.; Catalog #E7885) and mice are 8-wk-old male C57BL/6 mice (Charles River Laboratories). Animals are anesthetized and receive 20 μg of PPE in 50 μl of saline by surgical intra-tracheal instillation or 50 μl of saline alone (sham control group) on day 0. The day after PPE-instillation, the mice are randomly divided into two groups and receive daily administration by oral gavage of either test compound in water (final concentration 10 mg/kg qd, group designated “TC”), or water (vehicle control group) in a volume of 100 μL. The administration of compound or vehicle is continued for 3½ weeks. At the end of the experiment, animals are weighed and animals are sacrificed before determining arterial blood gas and isolation of lungs for histo-morphology and histo-immunology. Treatment measures include 1) effects on arterial oxygen levels. Arterial oxygen levels are an indicator of pulmonary function, and several studies have indicated reduced arterial oxygen in patients suffering from COPD and other pulmonary disorders (Celli B R, Cote C G, Lareau S C, Meek P M. Predictors of Survival in COPD: more than just the FEV1. Respir Med 2008 June; 102 Suppl 1:S27-S35). To evaluate the arterial oxygen pressure, blood samples are withdrawn from the abdominal artery and blood gas measurements were performed using a Siemens Rapidlab 248 blood gas analyzer. The arterial oxygen pressure in the test compound treated PPE-exposed animals is significantly higher than the pO₂ of vehicle treated animals. 2) To evaluate the effects of test compound on lung architecture, histomorphological analyses are carried out in H&E stained histological sections from paraffin embedded fixed lungs. The mean alveolar diameter is calculated by determining the mean linear intercept (Lm) from the analysis of 5 random fields in 6-10 lung slides in the different treatment groups. Typically, treatment with elastase results in an increase in alveolar diameter from an average of 42.5±1.6 in the sham operated animals to 56.5±5.8 in the elastase treated vehicle animals (Takahashi S, Nakamura H, Seki M et al. Reversal of elastase-induced pulmonary emphysema and promotion of alveolar epithelial cell proliferation by simvastatin in mice. Am J Physiol Lung Cell Mol Physiol 2008

May; 294(5):L882-L890; Plantier L, Marchand-Adam S, Antico V G et al. Keratinocyte growth factor protects against elastase-induced pulmonary emphysema in mice. Am J Physiol Lung Cell Mol Physiol 2007 November; 293(5):L 1230-L1239). Effective test compound will significantly decrease the mean alveolar intercept length (Lm) compared to vehicle treated PPE-exposed mice. This indicates a marked effect of TC on lung architecture.

Diabetes mellitus. 1. Compounds of the invention can reduce hyperglycemia in diabetic mice. Normal CD-1 mice are induced to develop hyperglycemia (diabetes) by i.v. injection with 100 mg/kg streptozotocin (STZ) followed by measurement of blood glucose in a week. The animals are treated with test compound at 2 mg/kg or vehicle daily starting the same day of STZ injection. Glucose samples are taken from the tail vein at day 7 with Ascensia ELITE blood glucose test strips (Bayer), and the blood glucose concentration is determined by glucose meters (Bayer). STZ induced diabetes, as shown by a significant increase in blood glucose levels compared to that in normal mice. Compounds of the invention can reduce blood glucose levels.

Muscular dystrophy. 1. In a genetic murine muscular dystrophy model, two months of intraperitoneal administration of a compound embodied herein can reduce the elevation in creatine kinase, indicating a beneficial effect on the disease.

Amyotrophic lateral sclerosis. 1. In SODG93A mouse model of ALS, daily compound administration starting at age 94 days (when neurofilament degeneration typically occurs) through day 122 can significantly improve hindlimb pathology score vs. In addition, a stride test can show improvement in treated animals. Survival of the treated animals can also be significantly (p<0.05) extended vs. vehicle-treated animals.

Ischemic limb disease. 1. Mouse hindlimb ischemia model. In a mouse hindlimb ischemia model treatment with a compound of the invention can produce greater recovery of hindlimb blow flow (as measured by laser Doppler imaging). Improved flux is associated with an increased number of capillaries in the ischemic muscle.

2. Hindlimb ischemia in non-obese diabetic (NOD) mice. In female NOD mice subjected to hindlimb ischemia, hindlimb blood flow (measured using a Laser Doppler imager) can demonstrate recovery by administration of a compound of the invention.

### Exemplary Formulas and Compounds

In one embodiment, compounds useful for the purposes described herein are represented by formula (I)

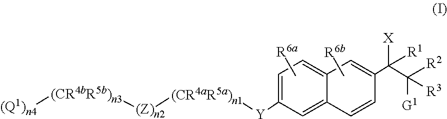

or an E or Z isomer thereof, syn or anti isomer thereof, an optically pure isomer thereof, or a pharmaceutically acceptable salt thereof, wherein:
X is an unsaturated heterocycle selected from pyrrolyl, pyrazolyl, imidazolyl, triazolyl, tetrazolyl, thiazole, or pyridinyl, any of which is optionally substituted with one or more independent R⁶⁶ substituents;
R¹ is a C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenyl carbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆amino carbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸²(R⁹)ₙ₅, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Y is an oxygen atom, sulfur atom, —(C═O)N(R⁷⁴)—, —CR^{4c}R^{5c} or —NR⁷⁴;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
Q¹ is C₀₋₆alkyl, —OR⁷⁵, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵ (R⁹⁵)ₙ₆, R⁷⁵ and R⁸⁵ taken together with the nitrogen atom to which they are attached form a 3-10 membered saturated ring, unsaturated ring, heterocyclic saturated ring, or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC1-10alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono (C₁₋₆alkyl)aminoC₁₋₆ alkyl, di(C₁₋₆ alkyl)aminoC₁₋₆ alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5C}, taken together with the respective carbon atom to which they are attached, form a carbonyl or 3-10 membered saturated or unsaturated ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated heterocyclic ring, wherein said ring is optionally substituted with R⁶⁹;
R^{6a}, R^{6b}, R⁶⁶, R⁶⁷, R⁶⁸, and R⁶⁹ are each independently halo, —OR—SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkyl C₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono (C₁₋₆alkyl)aminoC₁₋₆ alkyl, di(C₁₋₆alkyl)aminoC₁₋₆alkyl, mono(aryl)aminoC₁₋₆alkyl, di(aryl)aminoC₁₋₆ alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;

R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R85, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl (aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl)(C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; and

n1, n2, n3, n4, n5, n6, and n7 are each independently equal to 0, 1 or 2.

In an aspect of the present invention, a compound is represented by Formula I, or a pharmaceutically acceptable salt thereof, wherein X is an optionally substituted imidazolyl or optionally substituted triazolyl, and the other variables are as described above.

In an embodiment of this aspect, a compound is represented by Formula I, or a pharmaceutically acceptable salt thereof, wherein X is a substituted imidazolyl or substituted triazolyl; R′ is hydrogen; and the other variables are as described above.

In a second aspect of the present invention, a compound is represented by Formula I, or a pharmaceutically acceptable salt thereof, wherein Y is oxygen, and the other variables are as described above.

In an embodiment of this second aspect, compounds useful for the purposes described herein are represented by Formula IA:

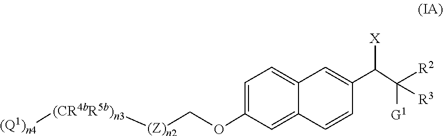

or an E or Z isomer thereof, syn or anti isomer thereof, an optically pure isomer thereof, or a pharmaceutically acceptable salt thereof, wherein:
X is an unsaturated heterocycle selected from pyrrolyl, pyrazolyl, imidazolyl, triazolyl, tetrazolyl, thiazole, or pyridinyl, any of which is optionally substituted with one or more independent R⁶⁶ substituents;
R² and R³ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀ alkyl carbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆-aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸² (R⁹)_{n≡}, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
Q¹ is C₀₋₆alkyl, —OR⁷⁵, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵ (R⁹⁵)ₙ₆, R⁷⁵ and R⁸⁵ taken together with the nitrogen atom to which they are attached form a 3-10 membered saturated ring, unsaturated ring, heterocyclic saturated ring, or heterocyclic unsaturated ring, any of which is optionally substituted with one or more independent halo, cyano, hydroxy, nitro, C1-10alkoxy, —SO₂N^{R6}R⁸⁶ or —NR⁷⁶R⁸⁶ substituents;
R^{4b} and R^{5b} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)aminoC₁₋₆ alkyl, di(C₁₋₆alkyl)aminoC₁₋₆ alkyl, mono(aryl)aminoC₁₋₆ alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or R^{4b} with R^{5b}, taken together with the respective carbon atom to which they are attached, form a carbonyl or 3-10 membered saturated or unsaturated ring, wherein said ring is optionally substituted with R⁶⁹; or R^{4b} with R^{5b}, taken together with the respective carbon atom to which they are attached, form a 3-10 membered saturated or unsaturated heterocyclic ring, wherein said ring is optionally substituted with R⁶⁹;
R⁶⁶, R⁶⁷, R⁶⁸, and R⁶⁹ are each independently —OR⁷⁸, —NR⁷⁷R⁸⁸(R⁹⁸),7, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono (C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸),7, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R85, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl)(C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; and
n2, n3, n4, n5, n6, and n7 are each independently equal to 0, 1 or 2.

In another embodiment of this second aspect, compounds useful for the purposes described herein are represented by Formula 1-B:

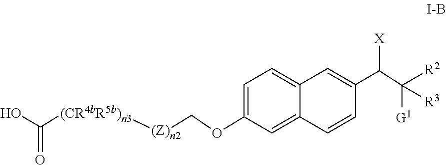

or an E or Z isomer thereof, syn or anti isomer thereof, an optically pure isomer thereof, or a pharmaceutically acceptable salt thereof, wherein:
X is substituted imidazolyl;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀ alkyl carbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀ alkyl, monoC₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR^{71R81}, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸² (R⁹)_{n≡}, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
R^{4b} and R^{5b} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀ alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono (C₁₋₆alkyl)aminoC₁₋₆ alkyl, di(C₁₋₆alkyl)aminoC₁₋₆ alkyl, mono(aryl)aminoC₁₋₆ alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or R^{4b} with R^{5b}, taken together with the respective carbon atom to which they are attached, form a carbonyl or 3-10 membered saturated or unsaturated ring, wherein said ring is optionally substituted with R⁶⁹;
R⁶⁷, R⁶⁸, and R⁶⁹ are each independently —OR⁷⁸, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono (C₁₋₆ alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆ alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl)(C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; and
n2, n3, n4, n5, n6, and n7 are each independently equal to 0, 1 or 2.

The compounds of the present invention include compounds represented by Formula I above, or a pharmaceutically acceptable salt thereof, and

1) wherein X is hetaryl, imidazolyl, or triazolyl, any of which is optionally substituted with one or more independent R⁶⁶ substituents; or
2) wherein X is imidazolyl or triazolyl; or
3) wherein X is hetaryl, imidazolyl, or triazolyl, any of which is optionally substituted with one or more independent R⁶⁶ substituents, and Q¹ is —CO₂H or —CO₂R⁷⁵; or
4) wherein Y is oxygen; or
5) wherein Y is oxygen and X is hetaryl, imidazolyl, or triazolyl, any of which is optionally substituted with one or more independent R⁶⁶ substituents; or
6) wherein Y is oxygen and X is imidazolyl or triazolyl; or
7) wherein Y is oxygen and X is imidazolyl or triazolyl and Q¹ is —CO₂H or —CO₂R⁷⁵; or
8) wherein Y is oxygen and R^{4a} and R^{5a} are each hydrogen; or
9) wherein X is hetaryl, imidazolyl, or triazolyl, any of which is optionally substituted with one or more independent R⁶⁶ substituents; R¹, R² and R³ are each independently C₀₋₁₀ alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁴R⁸⁵; R^{4a}; R^{4b}, R^{1a}, and R^{1b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a} or R^{4b} with R^{5b} taken together with the respective carbon atom to which they are attached form a 3-10 membered saturated or unsaturated heterocyclic ring, wherein said ring is optionally substituted with R⁶⁹; and, are each independently halo, —OR⁷⁸, CONR⁷⁸R⁸⁸, —NO₂, —CN, —S(O)ₙ₇R⁷⁸, —SO₂NR⁷⁸R⁸⁸, or C₀₋₁₀alkyl; or
10) wherein X is imidazolyl or triazolyl; R¹is hydrogen, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is —CO₂R⁷⁵ or —CONR⁷⁵R⁸⁵; R^{4a}, R^{4b}, R^{5a} and R^{5b} are each independently a C₀₋₁₀alkyl, any of which is optionally substituted with one or more independent halo, cyano, nitro, —OR, —SO₂ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; or
11) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4a} and R^{5a} are each hydrogen; R^{4b} and R^{5b} are each independently a C₀₋₁₀alkyl, any of which is optionally substituted with R⁶⁹; or R^{4b} with R^{5b} taken together with the respective carbon atom to which they are attached form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; or
12) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; or
13) wherein X is hetaryl, imidazolyl, or triazolyl, any of which is optionally substituted with one or more independent R⁶⁶ substituents; R¹, R² and R³ are each independently C₁₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀ alkyl; and R^{4b} with R^{5b} taken together with the respective carbon atom to which they are attached form a cyclopropyl, cyclobutyl, cyclopentyl, or cyclohexyl ring; or
14) wherein X is hetaryl, imidazolyl, or triazolyl, any of which is optionally substituted with one or more independent R⁶⁶ substituents; R¹, R² and R³ are each independently C₁₀₋₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR′, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; and R^{4b} and R^{5b} are both ethyl or are both methyl or are independently ethyl or methyl; or
15) wherein X is hetaryl, imidazolyl, or triazolyl, any of which is optionally substituted with one or more independent R⁶⁶ substituents; R¹, R² and R³ are each independently C₁₀₋₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; and Q¹is —CO₂R; or
16) wherein X is hetaryl, imidazolyl, or triazolyl, any of which is optionally substituted with one or more independent R⁶⁶ substituents; R¹, R² and R³ are each independently C₁₀₋₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; and Q¹is —CO₂H; or
17) wherein X is hetaryl, imidazolyl, or triazolyl, any of which is optionally substituted with one or more independent R⁶⁶ substituents; R¹, R² and R³ are each independently C₀₋₁₀alkyl; Y is oxygen; Q¹ is C₀₋₆alkyl, CO₂R⁷⁵, or —CoNR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and G¹ is di(C₁₋₆alkyl)amino; or
18) wherein X is hetaryl, imidazolyl, or triazolyl, any of which is optionally substituted with one or more independent R⁶⁶ substituents; R¹, R² and R³ are each independently C₀₋₁₀alkyl; Y is oxygen; Q¹ is C₀₋₆alkyl, CO₂R⁷⁵, or —CoNR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and G¹ is di(C₁₋₆alkyl)amino, ethylmethylamino, diethylamino, or isopropylmethylamino; or
19) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² and R³ are each independently hydrogen, methyl, or ethyl; or
20) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

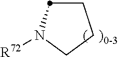

wherein , is the carbon to which they are attached;
or G¹ and R³ taken together with the carbon atom to which they are attached form

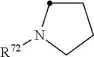

wherein is the carbon to which they are attached, any of which is optionally substituted by 1-10 independent R⁶⁷ substituents; or
21) wherein X is imidazole; or
22) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a} and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen and R³ is methyl; or
23) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen and R³ is ethyl; or
24) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² and R³ are both methyl; or
25) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen; and G¹ and R³ taken together with the carbon atom to which they are attached form

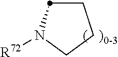

wherein is the carbon to which they are attached, or G¹ and R³ taken together with the carbon atom to which they are attached form

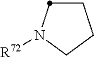

wherein is the carbon to which they are attached, any of which is optionally substituted by 1-10 independent R⁶⁷ substituents; and n2, n3, and n4 are each 1 and Z is aryl; or
26) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen; and G¹ and R³ taken together with the carbon atom to which they are attached form

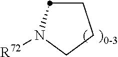

wherein is the carbon to which they are attached, or G¹ and R³ taken together with the carbon atom to which they are attached form

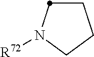

wherein is the carbon to which they are attached, any of which is optionally substituted by 1-10 independent R⁶⁷ substituents; n2 is 1; n3 and n4 are each 0; and Z is aryl; or
27) wherein Z is aryl or aryloxy or oxyaryl; or
28) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen; and G¹ and R³ taken together with the carbon atom to which they are attached form

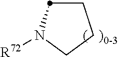

wherein is the carbon to which they are attached, or G¹ and R³ taken together with the carbon atom to which they are attached form

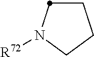

wherein is the carbon to which they are attached, any of which is optionally substituted by ₁₋₁₀ independent R⁶⁷ substituents; and n2, n3, and n4 are each 1 and Z is aryl; and n3 is 0; or
29) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen; and G¹ and R³ taken together with the carbon atom to which they are attached form

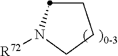

wherein is the carbon to which they are attached, or G¹ and R³ taken together with the carbon atom to which they are attached form

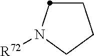

wherein is the carbon to which they are attached, any of which is optionally substituted by ₁₋₁₀ independent R⁶⁷ substituents; and n2, n3, and n4 are each 1 and Z is aryl; and n3 is 0; or
30) wherein X is imidazolyl or triazolyl; R¹ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is —CO₂R⁷⁵ or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; R² is hydrogen; and R³ is methyl; or
31) wherein X is imidazolyl or triazolyl; R¹ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is —CO₂R⁷⁵ or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; R² is hydrogen; and R³ is ethyl; or
32) wherein X is imidazolyl or triazolyl; R¹ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is —CO₂R⁷⁵ or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; and R² and R³ are methyl; or
33) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

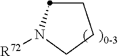

wherein is the carbon to which they are attached, or G¹ and R³ taken together with the carbon atom to which they are attached form

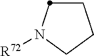

wherein is the carbon to which they are attached, any of which is optionally substituted by 1-10 independent R substituents; n1 and n2 are each 1; and Z is aryl; or
34) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

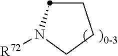

wherein is the carbon to which they are attached, or G¹ and R³ taken together with the carbon atom to which they are attached form

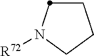

wherein is the carbon to which they are attached, any of which is optionally substituted by 1-10 independent R⁶⁷ substituents; n1 and n2 are each 1; n3 and n4 are each 0; and Z is aryl; or
35) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

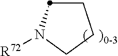

wherein is the carbon to which they are attached, or G¹ and R³ taken together with the carbon atom to which they are attached form

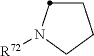

wherein is the carbon to which they are attached, any of which is optionally substituted by 1-10 independent R⁶⁷ substituents; n1 and n2 are each 1; Z is aryl; and Q¹ is —CO₂R⁷⁵; or
36) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

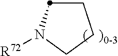

wherein is the carbon to which they are attached, or G¹ and R³ taken together with the carbon atom to which they are attached form

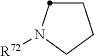

wherein is the carbon to which they are attached, any of which is optionally substituted by 1-10 independent R⁶⁷ substituents; n1 and n2 are each 1; Z is aryl; and Q¹ is —CO₂H; and wherein, in each case, the other variables are as defined above for Formula I.

The compounds useful for the purposes of the present invention include, by way of non-limiting examples: methyl 3-((6-(2-(dimethylamino)-1-(1H-imidazol-1-yl)propyl)naphthalen-2-yl)oxy)-2,2-dimethylpropanoate; methyl 2-(((6-(2-(dimethylamino)-1-(1H-imidazol-1-yl)propyl)naphthalen-2-yl)oxy)methyl)-2-ethylbutanoate; ethyl 1-(((6-(2-(dimethylamino)-1-(1H-imidazol-1-yl)propyl)naphthalen-2-yl)oxy)methyl)cyclopropanecarboxylate; ethyl 1-(((6-(2-(dimethylamino)-1-(1H-imidazol-1-yl)propyl)naphthalen-2-yl)oxy)methyl)cyclobutanecarboxylate ; methyl 1-(((6-(2-(dimethylamino)-1-(1H-imidazol-1-yl)propyl)naphthalen-2-yl)oxy)methyl)cyclopentanecarboxylate; methyl 1-(((6-(2-(dimethylamino)-1-(1H-imidazol-1-yl)propyl)naphthalen-2-yl)oxy)methyl)cyclohexanecarboxylate; methyl 1-(((6-(1-(1H-imidazol-1-yl)-2-(isopropyl(methyl)amino)propyl)naphthalen-2-yl)oxy)methyl)cyclopentanecarboxylate ; methyl 3-((6-(2-(diethylamino)-1-(1H-imidazol-1-yl)propyl)naphthalen-2-yl)oxy)-2,2-dimethylpropanoate; methyl 3-((6-(1-(1H-imidazol-1-yl)-2-(isopropyl(methyl)amino)propyl)naphthalen-2-yl)oxy)-2,2-dimethylpropanoate; methyl 3-((6-(2-(ethyl(methyl)amino)-1-(1H-imidazol-1-yl)propyl)naphthalen-2-yl)oxy)-2,2-dimethylpropanoate; methyl 3-((6-(2-(dimethylamino)-1-(1H-imidazol-1-yl)butyl)naphthalen-2-yl)oxy)-2,2-dimethylpropanoate; methyl 4-(((6-(2-(dimethylamino)-1-(1H-imidazol-1-yl)propyl)naphthalen-2-yl)oxy)methyl)benzoate; methyl 3-(((6-(2-(dimethylamino)-1-(1H-imidazol-1-yl)propyl)naphthalen-2-yl)oxy)methyl)benzoate; 3-[6-(2-dimethylamino-1-imidazol-1-yl-propyl)-naphthalen-2-yloxy]-2,2-dimethyl-propionic acid; 2-[6-(2-dimethylamino-1-imidazol-1-yl-propyl)-naphthalen-2-yloxymethyl]-2-ethyl-butyric acid; 1-[6-(2-dimethylamino-1-imidazol-1-yl-propyl)-naphthalen-2-yloxymethyl]-cyclopropanecarboxylic acid; [6-(2-dimethylamino-1-imidazol-1-yl-propyl)-naphthalen-2-yloxymethyl]-cyclobutanecarboxylic acid; [6-(2-dimethylamino-1-imidazol-1-yl-propyl)-naphthalen-2-yloxymethyl]-cyclopentanecarboxylic acid; 1-[6-(2-dimethylamino-1-imidazol-1-yl-propyl)-naphthalen-2-yloxymethyl]-cyclohexanecarboxylic acid; 1-{6-[1-imidazol-1-yl-2-(isopropylmethylamino)-propyl]-naphthalen-2-yloxymethyl}-cyclopentanecarboxylic acid; 3-[6-(2-diethylamino-1-imidazol-1-yl-propyl)-naphthalen-2-yloxy]-2,2-dimethyl-propionic acid; {6-[1-imidazol-1-yl-2-(isopropylmethylamino)-propyl]-naphthalen-2-yloxy}-2,2-dimethyl-propionic acid; 3-6-[2-(ethyl-methyl-amino)-1-imidazol-1-yl-propyl]-naphthalen-2-yloxy}-2,2-dimethyl-propionic acid; 3-[6-(2-dimethylamino-1-imidazol-1-yl-propyl)-naphthalen-2-yloxy]-2,2-dimethyl-propionamide; 3-[6-(2-dimethylamino-1-imidazol-1-yl-propyl)-naphthalen-2-yloxy]-2,2, n-trimethyl-propionamide; [6-(2-dimethyl amino-1-imidazol-1-yl-propyl)-naphthalen-2-yloxy]-2,2,N,N-tetramethyl-propionamide; 3-[6-(2-dimethylamino-1-imidazol-1-yl-butyl)-naphthalen-2-yloxy]-2,2-dimethyl-propionic acid; 4-[6-(2-dimethylamino-1-imidazol-1-yl-propyl)-naphthalen-2-yloxymethyl]-benzoic acid; 3-[6-(2-dimethylamino-1-imidazol-1-yl-propyl)-naphthalen-2-yloxymethyl]-benzoic acid; 4-[6-(2-dimethylamino-1-imidazol-1-yl-propyl)-naphthalen-2-yloxymethyl]-benzamide; 4-[6-(2-dimethylamino-1-imidazol-1-yl-propyl)-naphthalen-2-yloxymethyl]-n-methyl-benzamide; 4-[6-(2-dimethylamino-1-imidazol-1-yl-propyl)-naphthalen-2-yloxymethyl]-N,N-dimethyl-benzamide; and 1-[(6-benzyloxy-naphthalen-2-yl)-(1-methyl-pyrrolidin-2-yl)-methyl]-1H-imidazole.

In another embodiment, compounds useful for the purposes described herein are represented by formula (II)

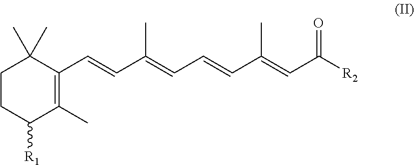

and E or Z isomers thereof, syn or anti isomers thereof, optically pure isomers thereof, or pharmaceutically acceptable salt thereof, and a pharmaceutically acceptable carrier therefor, wherein:
R¹ is an optionally substituted azole, sulfur, oxygen, nitrogen, pyridyl, acetylinic, cyclopropyl-amine, ester, oxime, cyano, amino, azido, cyclopropylamino, oxirane, aziridine, thiirane, thiol, alkylthiol, —OR₄ wherein R⁴ is hydrogen or an alkyl group, cyclopropylether, an oxygen containing group that forms, together with the 4-position carbon, an oxirane group; —NR⁵R⁶, where R⁵ and R⁶ are independently selected from the group consisting of hydrogen and alkyl groups, or R⁵ and R⁶ may together form a ring; and
R² is selected from the group consisting of hydroxyl, aminophenol, —OR³ and an azole group, wherein R³ is selected from the group consisting of alkyl, aryl and heterocyclic groups;
In certain embodiments, R¹ is selected from the group consisting of sulfur containing groups, oxygen containing groups, nitrogen containing groups, acetylinic, ester groups, oxime and aziridine; and R² is selected from the group consisting of hydroxyl, aminophenol, —OR³ and azole groups, wherein R₃ is selected from the group consisting of alkyl, aryl and heterocyclic groups.

In other embodiments, R₁ is optionally substituted azole, sulfur, oxygen, nitrogen, pyridyl, acetylinic, cyclopropyl-amine, esters, oxime, cyano, oxirane or aziridine; and R₂ is hydroxyl, an aminophenol, an ester, or an azole.

R₁ may be a sulfur containing group. Examples of such sulfur containing groups include thiirane, thiol and alkylthiol derivatives. Examples of such alkylthiol derivatives include C₁ to C₁₀ alkyl thiols.

R₁ may be an oxygen containing group. Examples of oxygen containing groups include —OR⁴, where R⁴ is hydrogen or an alkyl group (preferably a 1-10 carbon alkyl, more preferably methyl or ethyl), cyclopropylether or an oxygen containing group that forms, together with the 4-position carbon, an oxirane group.

R₁ may be a nitrogen containing group. Examples of such nitrogen containing groups include the formula —NR⁵R⁶, where R⁵ and R⁶ are independently selected from the group consisting of hydrogen and alkyl groups (preferably a 1-10 carbon alkyl, more preferably methyl or ethyl), or R⁵ and R⁶ may together form a ring. Preferably the ring formed by R⁵ and R⁶ is an imidazolyl ring or a triazole ring.

Preferable azole substituent groups include imidazoles and triazoles. More preferably, the azole substituent groups include 1H imidazole-1-yl, 1H 1,2,4-triazol-1-yl and 4H-1,2,4-triazol-4-yl.

R¹ may be a cyano, amino, azido, cyclopropylamino, or R¹ is a nitrogen containing group that forms, together with the 4-position carbon, an aziridine group or an oxime group.

R¹ may also be a pyridyl group or an allylic azole group, preferably methyleneazolyl.

The definitions for R¹ of an ester includes substituent groups that contain an ester moiety, including substituent groups attached via an ester moiety.

R² may be preferably selected from the group consisting of hydroxyl, aminophenol, —OR³ and azole groups, wherein R³ is selected from the group consisting of alkyl, aryl and heterocyclic groups, more preferably, hydroxyl or —OCH₃ (methoxy).

Said alkyl substituents for the above identified substituent groups include substituted and unsubstituted alkyl groups, branched and straight chain and cyclo alkyl groups, such as cyclopropyl.

The term “aryl” includes a phenyl or naphthyl ring.

Non-limiting examples of compounds of formula (II) useful for the purposes described herein include: (±)-4-(1H-imidazol-1-yl)-methyl retinoate, (±)-4-(1H-imidazole-1-yl)retinoic acid, (±)-4-(1H-1,2,4-triazol-1-yl)methyl retinoate, (±)-4-(4H-1,2,4-triazole-4-yl)methyl retinoate, (±)-4-(1H-1,2,4-triazol-1-yl)retinoic acid, and (±)-4-(4H-1,2,4-triazol-4-yl)retinoic acid.

Some of the foregoing compounds in formulas (I), (I-A), (I-B) and (II) can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of the invention for the uses described herein may be prepared by crystallization under different conditions and may exist as one or a combination of polymorphs of compound forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric forms, their stereoisomers, their polymorphs, their pharmaceutically acceptable salts their pharmaceutically acceptable solvates and pharmaceutically acceptable compositions containing them.

Compounds of this invention for the uses described include those specifically set forth above and described herein, and are illustrated in part by the various classes, subgenera and species disclosed elsewhere herein. Additionally, the present invention provides pharmaceutically acceptable derivatives of the inventive compounds, and methods of treating a subject using these compounds, pharmaceutical compositions thereof, or either of these in combination with one or more additional therapeutic agents. Certain compounds of the present invention are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. It will be appreciated that the compounds, as described herein, may be substituted with any number of substituents or functional moieties.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

### Pharmaceutical Compositions

As discussed above this invention provides uses of compounds described herein that have biological properties useful for the treatment of chronic obstructive pulmonary diseases.

Accordingly, in another aspect of the present invention, pharmaceutical compositions for the uses described herein are provided, which comprise any one or more of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved agent to treat the same or related indication, or it may be any one of a number of agents undergoing approval in the Food and Drug Administration that ultimately obtain approval for the treatment of any disorder related to CYP26 activity. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood, or N-demethylation of a compound of the invention. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. By way of example, N-methylated pro-drugs of pyrazoles embodied by the invention are embraced herein.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Formulations for intraocular administration are also included. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-inflammatory agent), or they may achieve different effects (e.g., control of any adverse effects). In non-limiting examples, one or more compounds of the invention may be formulated with at least one cytokine, growth factor or other biological, such as an interferon, e.g., alpha interferon, or with at least another small molecule compound. Non-limiting examples of pharmaceutical agents that may be combined therapeutically with compounds of the invention include: antivirals and antifibrotics such as interferon alpha, combination of interferon alpha and ribavirin, Lamivudine, Adefovir dipivoxil and interferon gamma; anticoagulants such as heparin and warfarin; antiplatelets e.g., aspirin, ticlopidine and clopidogrel; other growth factors involved in regeneration, e.g., VEGF and FGF and mimetics of these growth factors; antiapoptotic agents; and motility and morphogenic agents.

In certain embodiments, the pharmaceutical compositions of the present invention further comprise one or more additional therapeutically active ingredients (e.g., anti-inflammatory and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs.

### Research Uses, Clinical Uses, Pharmaceutical Uses and Methods of Treatment

In certain embodiments, the method involves the administration of a therapeutically effective amount of the compound or a pharmaceutically acceptable derivative thereof to a subject (including, but not limited to a human or animal) in need of it. Subjects for which the benefits of the compounds of the invention are intended for administration include, in addition to humans, livestock, domesticated, zoo and companion animals.

Based on the studies described herein, it has been found that inhibiting the activity of the CYP26 in vivo is an effective therapeutic approach to the treatment and prevention of a number of conditions and diseases as described herein by nonlimiting example. Any means of inhibiting CYP26 is embodied herein for the therapeutic purposes herein. Moreover, any condition, injury or disease modulated by the in-vivo level of all trans retenoic acid (ATRA) is encompassed herein. In certain embodiments, a compound or composition of the invention can be administered to inhibit CYP26 to increase endogenous ATRA, and an exogenous retinoic acid can be adminstered to further enhance the benefit of any of the conditions and diseases disclosed herein. It will be appreciated that the compounds and compositions, according to the method of the present invention, may be administered using any amount and any route of administration effective for the treatment of diseases in which inhibiting CYP26 activity or the activities thereof have a therapeutically useful role. Thus, the expression “effective amount” as used herein, refers to a sufficient amount of agent to modulate CYP26 activity (e g , inhibit CYP26 activity or increase endogenous ATRA), and to exhibit a therapeutic effect. The exact amount required will vary from subject to subject, depending on the species, age, and general condition of the subject, the severity of the infection, the particular therapeutic agent, its mode and/or route of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, subcutaneously, intradermally, intra-ocularly, topically (as by powders, ointments, or drops), buccally, as an oral or nasal spray, or the like, depending on the severity of the disease or disorder being treated. In certain embodiments of the invention, the compound or pharmaceutical composition of the invention is administered by a route and a dose and frequency of dosing to provide therapeutic levels to achieve the benefits described herein. Preferably routes of administration other than parenteral to address the lung diseases described herein include inhalation, such as by use of aerosols or fine powders, and other intra-pulmonary routes and methods. In certain embodiments, the small molecule compounds of the invention may be administered at dosage levels of about 0.001 mg/kg to about 50 mg/kg, preferably from about 0.1 mg/kg to about 10 mg/kg for parenteral administration, or preferably from about 1 mg/kg to about 50 mg/kg, more preferably from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 5₀₋₁₀0 mg/kg) can be administered to a subject. In certain embodiments, compounds are administered orally or parenterally.

Moreover, pharmaceutical compositions comprising one or more compounds of the invention may also contain other compounds or agents for which co-administration with the compound(s) of the invention is therapeutically advantageous. As many pharmaceutical agents are used in the treatment of the diseases and disorders for which the compounds of the invention are also beneficial, any may be formulated together for administration. Synergistic formulations are also embraced herein, where the combination of at least one compound of the invention and at least one other compounds act more beneficially than when each is given alone.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

Compounds useful for the purposes herein and as described above can be prepared following the guidance provided in WO2005/007631, based on PCT/US2004/022282, and in WO2002/03912, based on PCT/01/16524, both contents of which are incorporated herein by reference in their entireties. Furthermore, compounds described in the aforementioned documents are useful for the purposes herein.

### Example 1

### Use of CYP26 Inhibition in Emphysema

The efficacy of a compound embodied herein was evaluated in the porcine pancreatic elastase (PPE)-induced emphysema murine model. For the induction of emphysema, the protocol described in the literature by Takahashi and colleagues (Takahashi S, Nakamura H, Seki M et al. Reversal of elastase-induced pulmonary emphysema and promotion of alveolar epithelial cell proliferation by simvastatin in mice. Am J Physiol Lung Cell Mol Physiol 2008 May; 294(5):L882-L890) was followed. Porcine pancreatic elastase (PPE) was obtained from Sigma (St. Louis, Mo.; Catalog #E7885) and mice were 8-wk-old male C57BL/6 mice (Charles River Laboratories). Animals were anesthetized and received 20 μg of PPE in 50 μl of saline by surgical intra-tracheal instillation or 50 μA of saline alone (sham control group) on day 0. The day after PPE-instillation, the mice were randomly divided into two groups and received daily administration by oral gavage of either test compound in water (final concentration 10 mg/kg qd, group designated “TC”), or water (vehicle control group) in a volume of 100 μA. The administration of compound or vehicle was continued for 3½ weeks. At the end of the experiment, animals were weighed and animals were sacrificed before determining arterial blood gas and isolation of lungs for histo-morphology and histo-immunology.

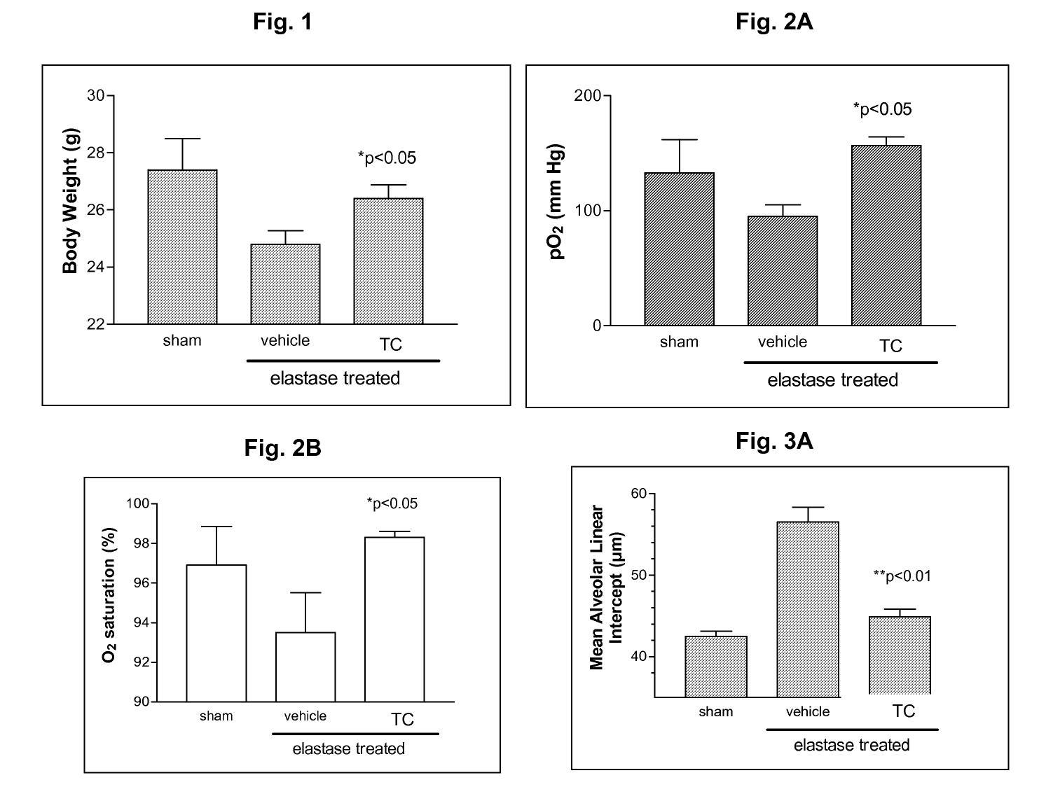

The elastase treatment in this study resulted in a reduction of the body weight (p<0.05) compared to sham operated age-matched control animals (FIG. 1). The weight of TC-treated animals, on the other hand, was significantly higher than that of the vehicle treated animals (p<0.05). A significant number of COPD patients have weight loss and a decreased body mass index is correlated with increased mortality (Celli B R, Cote C G, Marin J M et al. The body-mass index, airflow obstruction, dyspnea, and exercise capacity index in chronic obstructive pulmonary disease. N Engl J Med 2004 Mar. 4; 350(10):1005-12). The body mass index is a component of the BODE index (Body mass Index, Obstruction of airflow, Dyspnea, Excersice capability), which is capable of predicting COPD-related hospitalization and mortality more accurately than the individual components that make up this index (Celli B R, Cote C G, Lareau S C, Meek P M. Predictors of Survival in COPD: more than just the FEV1. Respir Med 2008 June; 102 Suppl 1:S27-S35). The positive effect of TC treatment on body weight may therefore be of clinical significance.

Treatment significantly affects arterial oxygen levels. Arterial oxygen levels are an indicator of pulmonary function, and several studies have indicated reduced arterial oxygen in patients suffering from COPD and other pulmonary disorders (Celli B R, Cote C G, Lareau S C, Meek P M. Predictors of Survival in COPD: more than just the FEV1. Respir Med 2008 June; 102 Suppl 1:S27-S35). To evaluate the arterial oxygen pressure, blood samples were withdrawn from the abdominal artery and blood gas measurements were performed using a Siemens Rapidlab 248 blood gas analyzer. The arterial oxygen pressure in the TC treated PPE-exposed animals was significantly higher than the pO₂ of vehicle treated animals (FIG. 2A; p<0.05) to a level that was similar to the sham control animals. This reflects similar changes in O₂ saturation of the blood (FIG. 2B).

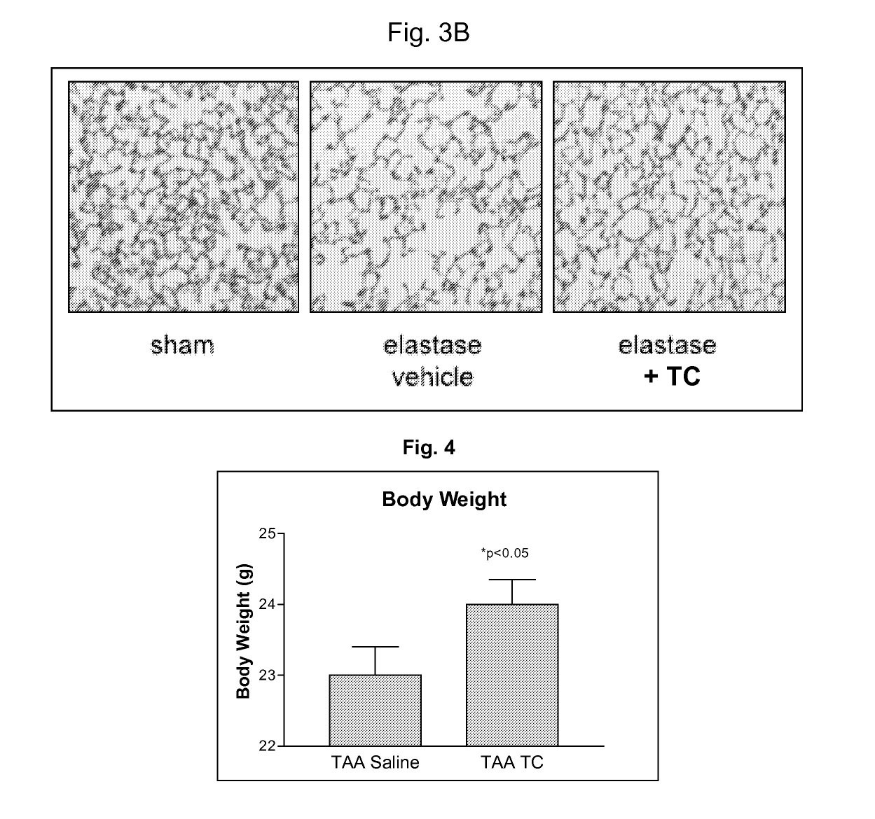

TC ameliorates PPE-induced lung damage. To evaluate the effects of TC on lung architecture, histomorphological analyses were carried out in H&E stained histological sections from paraffin embedded fixed lungs. The mean alveolar diameter was calculated by determining the mean linear intercept (Lm) from the analysis of 5 random fields in 6-10 lung slides in the different treatment groups (for sham n=6 and for vehicle and TC groups, n=10). As expected, the treatment with elastase resulted in an increase in alveolar diameter from an average of 42.5±1.6 in the sham operated animals to 56.5±5.8 in the elastase treated vehicle animals (FIG. 3A).

This increase in alveolar diameter as a result of elastase treatment is similar to that observed by others that used this or similar murine models (Takahashi S, Nakamura H, Seki M et al. Reversal of elastase-induced pulmonary emphysema and promotion of alveolar epithelial cell proliferation by simvastatin in mice (Am J Physiol Lung Cell Mol Physiol 2008 May; 294(5):L882-L890; Plantier L, Marchand-Adam S, Antico V G et al. Keratinocyte growth factor protects against elastase-induced pulmonary emphysema in mice. Am J Physiol Lung Cell Mol Physiol 2007 November; 293(5):L1230-L1239). However, as shown below in the HE-stained sections and in the quantified histomorphological alveolar diameter (FIG. 3B), TC significantly decreased the mean alveolar intercept length (Lm; p<0.01) compared to vehicle treated PPE-exposed mice. This indicates a marked effect of TC on lung architecture.

### Example 2

### Use of CYP26 Inhibition in Liver Fibrosis

The efficacy of a compound embodied herein was evaluated in the thioacetamide (TAA)-induced murine model of liver fibrosis. For the induction of liver fibrosis, TAA was administered at 200 mg/kg, i.p., 3 times a week (Mon, Wed, and Fri) for 4 weeks. Starting the day after the first TAA administration, mice were treated with test compound at 10 mg/kg p.o. in a saline vehicle, 5 times a week (Mon-Fri) for 4 weeks. Control groups were not treated with TAA, or were treated with TAA and vehicle.

After four weeks, the TAA treatment in this study resulted in a reduction of the body weight compared to age-matched control animals. The weight of test compound treated animals was significantly higher than that of the vehicle treated animals (p<0.05). The positive effect of test compound treatment on body weight suggested a beneficial effect of the compound on the overall health of the animals (FIG. 4).

TC ameliorates TAA-induced elevations in serum AST and ALT. Increases in serum liver transaminases AST and ALT are well-established markers of hepatic dysfunction (Lott R S, Helmboldt K M, Madaras-Kelly K J. Retrospective evaluation of the effect of valproate therapy on transaminase elevations in patients with hepatitis C. Pharmacotherapy 2001 November; 21(11):1345-51; Carlson M K, Gleason P P, Sen S. Elevation of hepatic transaminases after enoxaparin use: case report and review of unfractionated and low-molecular-weight heparin-induced hepatotoxicity. Pharmacotherapy 2001 January; 21(1): 108-13.; Balkan J, Dogru-Abbasoglu S, Kanbagli O, Cevikbas U, ykac-Toker G, Uysal M. Taurine has a protective effect against thioacetamide-induced liver cirrhosis by decreasing oxidative stress. Hum Exp Toxicol 2001 May; 20(5):251-4). In normal, untreated mice, serum AST values are 53 U/L and

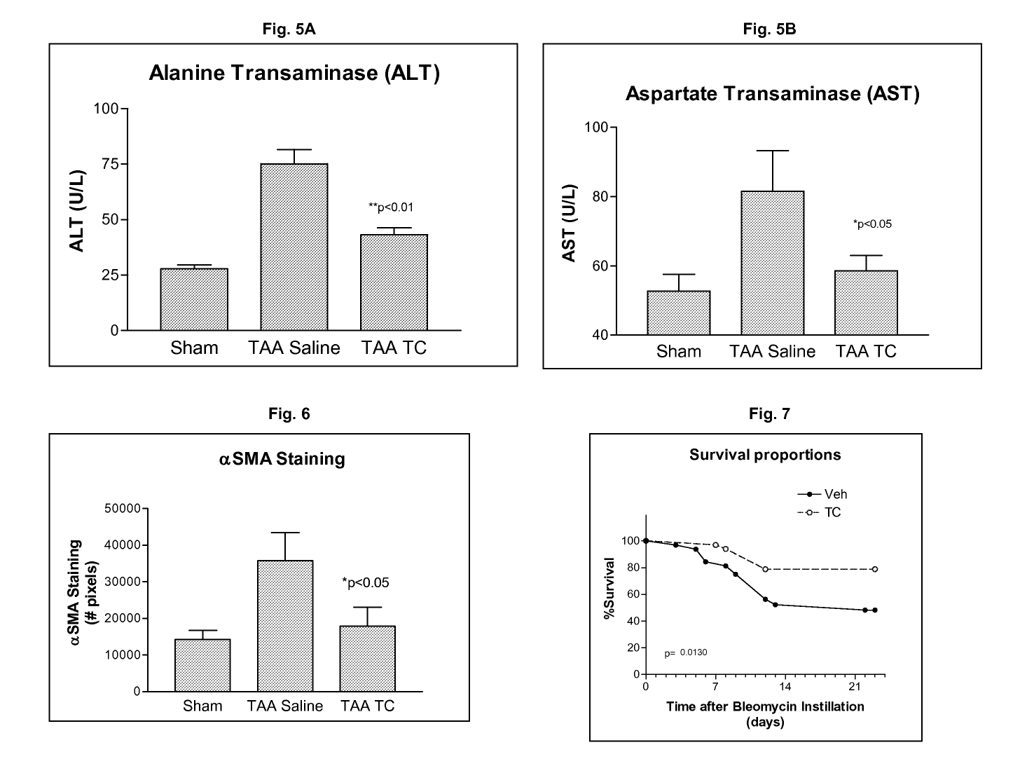

ALT values average 28 U/L. TAA treatment resulted in marked increases in serum AST (approximately 2-fold) and ALT levels (approximately 3-fold). However treatment with TC significantly reduced the hepatic dysfunction in these animals (n≧12 animals/group). ANOVA analysis followed by Bonferroni's post-test indicated a p<0.01 for ALT and p<0.05 for AST levels, comparing the vehicle and TC treated TAA exposed animals (FIGS. 5A & 5B).

TC ameliorates TAA-induced elevations in αSMA expression. Alpha smooth muscle actin (αSMA) is an important fibrotic marker protein expressed in response to various cytokines and fibrosis-inducing challenges. To determine the effect of TC treatment on αSMA expression in vivo, quantitative immunohistochemistry on section from normal, TAA-exposed animals and TAA exposed, TC treated animals were performed. A mouse monoclonal antibody (Sigma 5691) for the immuno histochemical detection of αSMA was used. As expected, TAA resulted in a marked elevation in αSMA expression. However, TAA-exposed animals that were treated with TC had significantly reduced αSMA expression compared to TAA treated animals (FIG. 6). An increase in αSMA positive cells is associated with the progression of hepatic fibrosis in the liver of patients with alcoholic liver disease and that the appearance of αSMA in liver mesenchymal cells is closely related to the process of hepatic fibrosis in both rat and man (Nouchi T, Tanaka Y, Tsukada T, Sato C, Marumo F. Appearance of alpha-smooth-muscle-actin-positive cells in hepatic fibrosis. Liver 1991 April; 11(2): 100-5; Yamaoka K, Nouchi T, Marumo F, Sato C. Alpha-smooth-muscle actin expression in normal and fibrotic human livers. Dig Dis Sci 1993 August; 38(8):1473-9). The marked effect of TC treatment on the expression of the fibrotic biomarker αSMA strongly indicates that TC has activity to prevent TAA-induced liver fibrosis.

### Example 3

### Use of CYP26 Inhibition in Lung Fibrosis

Efficacy of TC in ameliorating bleomycin-induced pulmonary fibrosis. For the induction of lung fibrosis, 3 U/kg bleomycin (Sigma, St. Louis, Mo., Catalog #B5507) or 50 μl of saline alone (sham control group) on day 0 was intra-tracheally instilled. The day after bleomycin instillation, mice were randomly divided into two groups and received daily administration by oral gavage of either TC in water or water (vehicle control group) in a volume of 100 μL. Two experiments were conducted: in experiment 1, 5 and 10 mg/kg dose was used and in experiment 2 the treatment was performed with 10 mg/kg qd. The administration was continued for 2-3 weeks.

TC improves survival. Instillation of bleomycin resulted in a significant mortality 2-3 weeks after the operation. However, bleomycin-exposed animals that received daily oral doses of 10 mg/kg TC had significantly improved survival compared to vehicle treated controls. Combining the survival data from two separate experiments (total number of vehicle treated animals was 29 and from TC treated animals was 32) and analyzing the survival by Kaplan-Meier analysis resulted in p=0.013 (FIG. 7).

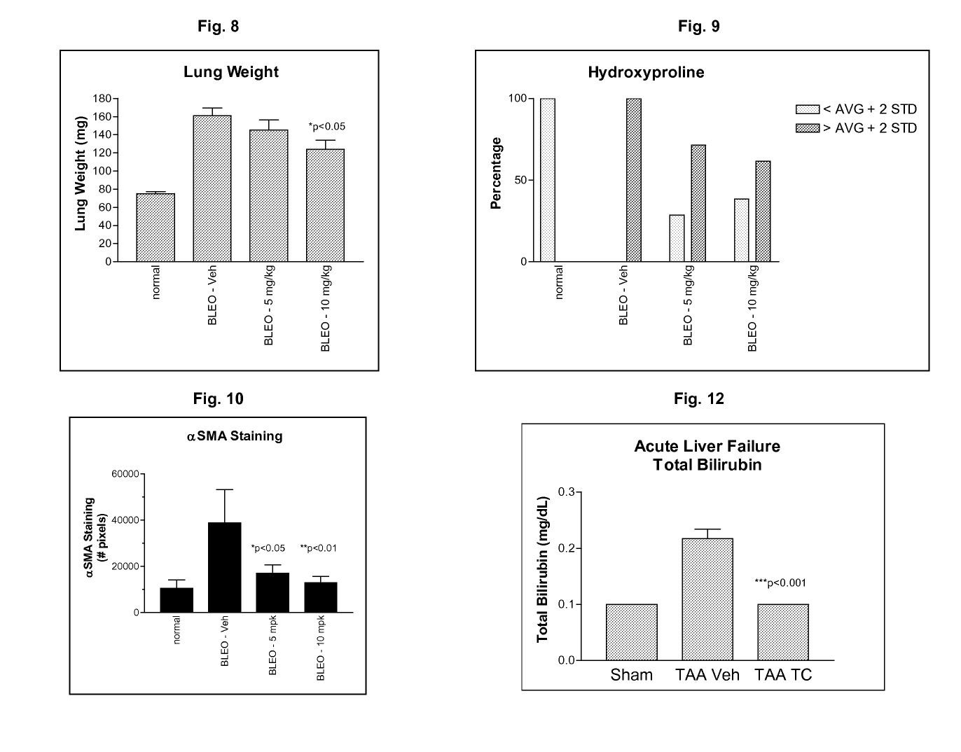

TC improves lung weight. A single intra-tracheal instillation of bleomycin resulted in a marked increase in lung weight in the vehicle-treated animals, presumably as a result of significant pulmonary infiltration and of induction of fibrosis. However, bleomycin-exposed animals that received daily oral doses of TC showed a dose dependent smaller increase in lung weight. The 10 mg/kg group of experiment 1 showed a p<0.05 by one-way analysis of variance followed by Bonferroni's post-test. Combining the lung weights from the two separate studies, p<0.001 by on-way analysis of variance followed by Bonferroni's post-test (FIG. 8).

TC reduces pulmonary hydroxyproline content. The quantification of pulmonary collagen was performed by determining the hydroxyproline content on the right lungs. The hydroxyproline content was markedly elevated in lungs from animals treated with bleomycin compared to non-treated controls. While all normal animals had a hydroxyproline content below the average+2 standard deviations (“normal” levels), all the bleomycin-treated animals had a pulmonary hydroxyproline content greater than the normal average+2 standard deviations (“fibrotic” hydroxyproline levels). Animals treated with bleomycin and TC at 5 or 10 mg/kg, however, showed a markedly increased proportion of animals with a “normal” hydroxyproline content (p<0.01). These data indicate that TC has strong activity to prevent bleomycin-induced pulmonary fibrosis in mice (FIG. 9).

TC reduces pulmonary bleomycin-induced αSMA expression. To determine the effect of TC treatment on αSMA expression in vivo, quantitative immunohistochemistry was performed on sections from study 1. A mouse monoclonal antibody (Sigma 5691) was used for the immuno histochemical detection of αSMA. As expected, bleomycin resulted in a marked elevation in αSMA expression in vehicle treated animals. However, bleomycin-exposed animals that were treated with TC had significantly reduced αSMA expression compared to the vehicle controls. ANOVA followed by Newman-Keuls posttest indicated a p<0.05 for the 5 mg/kg dose group and p<0.01 for the 10 mg/kg dose group (FIG. 10).

### Example 4

### Use of CYP26 Inhibition in Acute Liver Failure

Acute liver failure (ALF) is a clinical syndrome that is characterized by marked increases in serum aspartate amino transferase (AST) levels, alanine transferase (ALT) levels, nausea, vomiting, confusion and coma. Treatment of ALF involves hospitalization and often requires admission to the intensive care unit, where patients are closely monitored and supportive treatment is provided (Viana C F, Rocha T D, Cavalcante F P, Valenca Jr J T, Coelho G R, Garcia J H. Liver transplantation for acute liver failure: a 5 years experience. Arq Gastroenterol 2008 July; 45(3):192-4; Prasad K R, Lodge J P. ABC of diseases of liver, pancreas, and biliary system: Transplantation of the liver and pancreas. BMJ 2001 Apr. 7; 322(7290):845-7; Stravitz R T. Critical management decisions in patients with acute liver failure. Chest 2008 November; 134(5): 1092-102; Larson A M. Diagnosis and management of acute liver failure. Curr Opin Gastroenterol 2010 Mar. 6). Intravenous N-acetylcysteine has been found to be beneficial in both acetaminophen toxicity and non-acetaminophen-related acute liver failure (Koch A, Trautwein C. N-acetylcysteine on its way to a broader application in patients with acute liver failure. Hepatology 2010 January; 51(1):338-40; Sotelo N, de los Angeles D M, Gonzalez A, Dhanakotti N. Early treatment with N-acetylcysteine in children with acute liver failure secondary to hepatitis A. Ann Hepatol 2009 October; 8(4):353-8; Saito C, Zwingmann C, Jaeschke H. Novel mechanisms of protection against acetaminophen hepatotoxicity in mice by glutathione and N-acetylcysteine. Hepatology 2010 January; 51(1):246-54; Mumtaz K, Azam Z, Hamid S et al. Role of N-acetylcysteine in adults with non-acetaminophen-induced acute liver failure in a center without the facility of liver transplantation. Hepatol Int 2009 Aug. 29; Lee W M, Hynan L S, Rossaro L et al. Intravenous N-acetylcysteine improves transplant-free survival in early stage non-acetaminophen acute liver failure. Gastroenterology 2009 September; 137(3):856-64, 864; Ritter C, Reinke A, Andrades M et al. Protective effect of N-acetylcysteine and deferoxamine on carbon tetrachloride-induced acute hepatic failure in rats. Crit Care Med 2004 October; 32(10):2079-83; Sklar G E, Subramaniam M. Acetylcysteine treatment for non-acetaminophen-induced acute liver failure. Ann Pharmacother 2004 March; 38(3):498-500; Clark J. Acetaminophen poisoning and the use of intravenous N-acetylcysteine. Air Med J 2001 July; 20(4):16-7). While many people who develop acute liver failure recover with such care, liver transplantation is often required in people who continue to deteriorate or have adverse prognostic factors.

To test the effects of inhibition of CYP26 on the development of acute liver failure, TC was tested in the TAA-induced murine model of acute liver failure. For the induction of acute liver failure, we administered TAA at 200 mg/kg, ip, for three consecutive days. On the same day but prior to the first TAA administration, mice were treated with TC at 10 mg/kg po in a saline vehicle and animals were also treated with compound in the morning of day 4 before sacrifice. Control groups were not treated with TAA, or were treated with TAA and vehicle.

TC ameliorates TAA-induced elevations in serum AST and ALT. Increases in serum liver transaminases AST and ALT are well-established markers of hepatic dysfunction (Lott R S, Helmboldt K M, Madaras-Kelly K J. Retrospective evaluation of the effect of valproate therapy on transaminase elevations in patients with hepatitis C. Pharmacotherapy 2001 November; 21(11):1345-51; Carlson M K, Gleason P P, Sen S. Elevation of hepatic transaminases after enoxaparin use: case report and review of unfractionated and low-molecular-weight heparin-induced hepatotoxicity. Pharmacotherapy 2001 January; 21(1):108-13; Balkan J, Dogru-Abbasoglu S, Kanbagli O, Cevikbas U, ykac-Toker G, Uysal M. Taurine has a protective effect against thioacetamide-induced liver cirrhosis by decreasing oxidative stress. Hum Exp Toxicol 2001 May; 20(5):251-4). TAA treatment for three consecutive days resulted in dramatic increases in serum AST (approximately 25-fold) and ALT levels (approximately 70-fold). However treatment with TC significantly reduced the hepatic dysfunction in these animals (n≧12 animals/group). ANOVA analysis followed by Bonferroni's post-test indicated a p<0.001 for ALT and p<0.01 for AST levels, comparing the vehicle and TC treated TAA exposed animals (FIGS. 11A & 11B).

TC ameliorates TAA-induced elevations in serum bilirubin. An increase in serum bilirubin is another well-established marker of hepatic dysfunction (Lott R S, Helmboldt K M, Madaras-Kelly K J. Retrospective evaluation of the effect of valproate therapy on transaminase elevations in patients with hepatitis C. Pharmacotherapy 2001 November; 21(11):1345-51; Carlson M K, Gleason P P, Sen S. Elevation of hepatic transaminases after enoxaparin use: case report and review of unfractionated and low-molecular-weight heparin-induced hepatotoxicity. Pharmacotherapy 2001 January; 21(1):108-13; Balkan J, Dogru-Abbasoglu S, Kanbagli O, Cevikbas U, ykac-Toker G, Uysal M. Taurine has a protective effect against thioacetamide-induced liver cirrhosis by decreasing oxidative stress. Hum Exp Toxicol 2001 May; 20(5):251-4). TAA treatment resulted in a significant increase in total serum bilirubin levels (approximately 2-fold). However treatment with TC significantly reduced this increase. ANOVA analysis followed by Bonferroni's post-test indicated a p<0.001, comparing the vehicle and TC treated TAA exposed animals (FIG. 12).

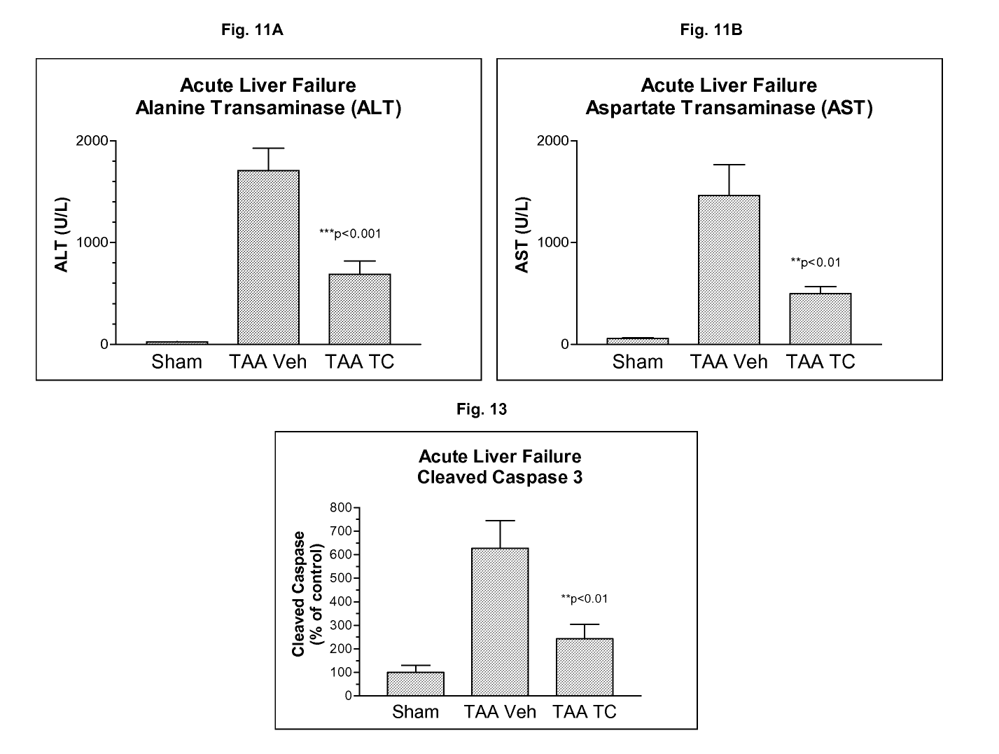

TC ameliorates TAA-induced elevations liver cell apoptosis. Cysteine dependent aspartate-directed proteases (caspases) play essential roles in programmed cell death (apoptosis). In cells undergoing apoptosis certain caspases, including caspase 3, are cleaved and activated. Acute liver failure is associated with massive hepatic apoptosis and immunohistochemical analysis of liver tissue sections from TAA exposed mice indicate that TAA administration in mice leads to a marked increase in cleaved caspase 3, an indicator of marked induction of apoptosis. However, treatment with TC significantly reduced this increase. ANOVA analysis followed by Bonferroni's post-test indicated a p<0.01, comparing the vehicle and TC treated TAA exposed animals (FIG. 13).

What is claimed is:

1. A method for treating a chronic obstructive pulmonary disease comprising administering to a subject in need thereof an effective amount of a pharmaceutical composition comprising a compound that inhibits cytochrome P450RA1 or CYP26.

2. A method of prevention, treatment or lessening of the severity of a condition or disease associated with or characterized by increased, excessive or inappropriate fibrosis comprising administering to a subject in need thereof an effective amount of a pharmaceutical composition comprising a compound that inhibits cytochrome P450RA1 or CYP26.

3. The method of prevention, treatment or lessening of the severity of a condition or disease associated with or characterized by ischemia-reperfusion injury, cerebral infarction, ischemic heart disease, acute liver failure, acute lung injury or renal disease comprising administering to a subject in need thereof an effective amount of a pharmaceutical composition comprising a compound that inhibits cytochrome P450RA1 or CYP26.

4. The method of any one of claims 1-3 wherein the compound has a structure represented by formula (I): 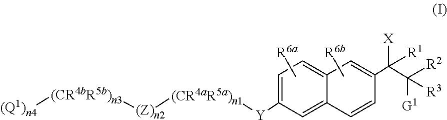
or an E or Z isomer thereof, syn or anti isomer thereof, an optically pure isomer thereof, or a pharmaceutically acceptable salt thereof, wherein:
X is an unsaturated heterocycle selected from pyrrolyl, pyrazolyl, imidazolyl, triazolyl, tetrazolyl, thiazole, or pyridinyl, any of which is optionally substituted with one or more independent R⁶⁶ substituents;
R¹ is a C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆ alkylaminocarbonyl, diC₁₋₆ amino carbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸² (R⁹)_{n≡}, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Y is an oxygen atom, sulfur atom, —(C═O)N(R⁷⁴)—, —CR^{4c}R^{5c} or —NR⁷⁴;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
Q¹ is C₀₋₆alkyl, —OR⁷⁵, —NR⁷⁵R⁸⁵(R⁹⁵).6, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵ (R⁹⁵)ₙ₆, R⁷⁵ and R⁸⁵ taken together with the nitrogen atom to which they are attached form a 3-10 membered saturated ring, unsaturated ring, heterocyclic saturated ring, or heterocyclic unsaturated ring, any of which is optionally substituted with one or more independent halo, cyano, hydroxy, nitro, C1-10alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono (C₁₋₆ alkyl)aminoC₁₋₆ alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated heterocyclic ring, wherein said ring is optionally substituted with R⁶⁹;
R^{6a}, R^{6b}, R⁶⁶, R⁶⁷, R⁶⁸, and R⁶⁹ are each independently halo, —OR—SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl , cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono (C₁₋₆alkyl)aminoC₁₋₆ alkyl, di(C₁₋₆alkyl)aminoC₁₋₆alkyl, mono(aryl)aminoC₁₋₆alkyl, di(aryl)aminoC₁₋₆alkyl, —N(C₁₋₆ alkyl)-C₁₋₆ alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R85, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈ alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylamino carbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄ alkyl) or —N(C₀₋₄alkyl)(C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀ alkynyl, —COOH, C₁₋₄alkoxycarbonyl, —CON(C₀₋₄ alkyl)(C₀₋₄ alkyl), —SO₂N(C₀₋₄alkyl)(C₀₋₄alkyl) or —N(C₀₋₄alkyl)(C₀₋₄alkyl) substituents; and
n1, n2, n3, n4, n5, n6, and n7 are each independently equal to 0, 1 or 2.

5. The method of any one of claims 1-4 wherein the compound is methyl 3-((6-(2-(dimethylamino)-1-(1H-imidazol-1-yl)propyl)naphthalen-2-yl)oxy)-2,2-dimethylpropanoate; methyl 2-(((6-(2-(dimethylamino)-1-(1H-imidazol-1-yl)propyl)naphthalen-2-yl)oxy)methyl)-2-ethylbutanoate; ethyl 1-(((6-(2-(dimethylamino)-1-(1H-imidazol-1-yl)propyl)naphthalen-2-yl)oxy)methyl)cyclopropanecarboxylate; ethyl 1-(((6-(2-(dimethylamino)-1-(1H-imidazol-1-yl)propyl)naphthalen-2-yl)oxy)methyl)cyclobutanecarboxylate; methyl 1-(((6-(2-(dimethylamino)-1-(1H-imidazol-1-yl)propyl)naphthalen-2-yl)oxy)methyl)cyclopentanecarboxylate; methyl 1-(((6-(2-(dimethylamino)-1-(1H-imidazol-1-yl)propyl)naphthalen-2-yl)oxy)methyl)cyclohexanecarboxylate; methyl 1-(((6-(1-(1H-imidazol-1-yl)-2-(isopropyl(methyl)amino)propyl)naphthalen-2-yl)oxy)methyl)cyclopentanecarboxylate; methyl 3-((6-(2-(diethylamino)-1-(1H-imidazol-1-yl)propyl)naphthalen-2-yl)oxy)-2,2-dimethylpropanoate; methyl 3-((6-(1-(1H-imidazol-1-yl)-2-(isopropyl(methyl)amino)propyl)naphthalen-2-yl)oxy)-2,2-dimethylpropanoate; methyl 3-((6-(2-(ethyl(methyl)amino)-1-(1H-imidazol-1-yl)propyl)naphthalen-2-yl)oxy)-2,2-dimethylpropanoate; methyl 3-((6-(2-(dimethylamino)-1-(1H-imidazol-1-yl)butyl)naphthalen-2-yl)oxy)-2,2-dimethylpropanoate; methyl 4-(((6-(2-(dimethylamino)-1-(1H-imidazol-1-yl)propyl)naphthalen-2-yl)oxy)methyl)benzoate; methyl 3-(((6-(2-(dimethylamino)-1-(1H-imidazol-1-yl)propyl)naphthalen-2-yl)oxy)methyl)benzoate; 3-[6-(2-dimethylamino-1-imidazol-1-yl-propyl)-naphthalen-2-yloxy]-2,2-dimethyl-propionic acid; 2-[6-(2-dimethylamino-1-imidazol-1-yl-propyl)-naphthalen-2-yloxymethyl]-2-ethyl-butyric acid; 1-[6-(2-dimethylamino-1-imidazol-1-yl-propyl)-naphthalen-2-yloxymethyl]-cyclopropanecarboxylic acid; [6-(2-dimethylamino-1-imidazol-1-yl-propyl)-naphthalen-2-yloxymethyl]-cyclobutanecarboxylic acid; [6-(2-dimethylamino-1-imidazol-1-yl-propyl)-naphthalen-2-yloxymethyl]-cyclopentanecarboxylic acid; 1-[6-(2-dimethylamino-1-imidazol-1-yl-propyl)-naphthalen-2-yloxymethyl]-cyclohexanecarboxylic acid; 1-{6-[1-Imidazol-1-yl-2-(isopropylmethylamino)-propyl]-naphthalen-2-yloxymethyl}-cyclopentanecarboxylic acid; 3-[6-(2-Diethylamino-1-imidazol-1-yl-propyl)-naphthalen-2-yloxy]-2,2-dimethyl-propionic acid; {6-[1-imidazol-1-yl-2-(isopropylmethylamino)-propyl]-naphthalen-2-yloxy}-2,2-dimethyl-propionic acid; 3-6-[2-(Ethyl-methyl-amino)-1-imidazol-1-yl-propyl]-naphthalen-2-yloxy}-2,2-dimethyl-propionic acid; 3-[6-(2-Dimethylamino-1-imidazol-1-yl-propyl)-naphthalen-2-yloxy]-2,2-dimethyl-propionamide ; 3-[6-(2-Dimethylamino-1-imidazol-1-yl-propyl)-naphthalen-2-yloxy]-2,2, N-trimethyl-propionamide ; 3-[6-(2-dimethylamino-1-imidazol-1-yl-propyl)-naphthalen-2-yloxy]-2,2,N,N-tetramethyl-propionamide; 3-[6-(2-Dimethylamino-1-imidazol-1-yl-butyl)-naphthalen-2-yloxy]-2,2-dimethyl-propionic acid; 4-[6-(2-Dimethylamino-1-imidazol-1-yl-propyl)-naphthalen-2-yloxymethyl]-benzoic acid; 3-[6-(2-Dimethylamino-1-imidazol-1-yl-propyl)-naphthalen-2-yloxymethyl]-benzoic acid; 4-[6-(2-Dimethylamino-1-imidazol-1-yl-propyl)-naphthalen-2-yloxymethyl]-benzamide; 4-[6-(2-Dimethylamino-1-imidazol-1-yl-propyl)-naphthalen-2-yloxymethyl]-N-methyl-benzamide; 4-[6-(2-dimethylamino-1-imidazol-1-yl-propyl)-naphthalen-2-yloxymethyl]-N,N-dimethyl-benzamide; or 1-[(6-Benzyloxy-naphthalen-2-yl)-(1-methyl-pyrrolidin-2-yl)-methyl]-1H-imidazole.

6. The method of any one of claims 1-3 wherein the compound has a structure represented by formula (I-A): 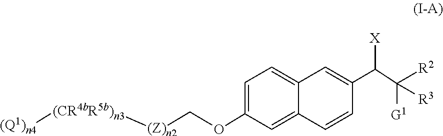
or an E or Z isomer thereof, syn or anti isomer thereof, an optically pure isomer thereof, or a pharmaceutically acceptable salt thereof, wherein:
X is an unsaturated heterocycle selected from pyrrolyl, pyrazolyl, imidazolyl, triazolyl, tetrazolyl, thiazole, or pyridinyl, any of which is optionally substituted with one or more independent R⁶⁶ substituents;
R² and R³ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆ alkylamino carbonyl, diC₁₋₆ amino carbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸²(R⁹)ₙ₅, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
Q¹ is C₀₋₆alkyl, —OR⁷⁵, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵ (R⁹⁵)ₙ₆, R⁷⁵ and R⁸⁵ taken together with the nitrogen atom to which they are attached form a 3-10 membered saturated ring, unsaturated ring, heterocyclic saturated ring, or heterocyclic unsaturated ring, any of which is optionally substituted with one or more independent halo, cyano, hydroxy, nitro, C1-10alkoxy, —SO₂N^{R6}R⁸⁶ or —NR⁷⁶R⁸⁶ substituents;
R^{4b} and R^{5b} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C1-10alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC1-10alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono (C₁₋₆alkyl)aminoC₁₋₆alkyl, di(C₁₋₆ alkyl)aminoC₁₋₆ alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or Rob with R^{5b}, taken together with the respective carbon atom to which they are attached, form a carbonyl or 3-10 membered saturated or unsaturated ring, wherein said ring is optionally substituted with R⁶⁹; or R^{4b} with R^{5b}, taken together with the respective carbon atom to which they are attached, form a 3-10 membered saturated or unsaturated heterocyclic ring, wherein said ring is optionally substituted with R⁶⁹;
R⁶⁶, R⁶⁷, R⁶⁸, and R⁶⁹ are each independently —OR⁷⁸, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono (C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆ alkyl, —N(C₁₋₆ alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R85, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl , cycloC₃₋₈alkenylC₁₋₁₀alkyl, cycloC₃₋alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄ alkyl) or —N(C₀₋₄alkyl)(C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; and
n2, n3, n4, n5, n6, and n7 are each independently equal to 0, 1 or 2.

7. The method of any one of claims 1-6 wherein the compound is methyl 3-((6-(2-(dimethylamino)-1-(1H-imidazol-1-yl)propyl)naphthalen-2-yl)oxy)-2,2-dimethylpropanoate; methyl 2-(((6-(2-(dimethylamino)-1-(1H-imidazol-1-yl)propyl)naphthalen-2-yl)oxy)methyl)-2-ethylbutanoate; ethyl 1-(((6-(2-(dimethylamino)-1-(1H-imidazol-1-yl)propyl)naphthalen-2-yl)oxy)methyl)cyclopropanecarboxylate; ethyl 1-(((6-(2-(dimethylamino)-1-(1H-imidazol-1-yl)propyl)naphthalen-2-yl)oxy)methyl)cyclobutanecarboxylate; methyl 1-(((6-(2-(dimethylamino)-1-(1H-imidazol-1-yl)propyl)naphthalen-2-yl)oxy)methyl)cyclopentanecarboxylate; methyl 1-(((6-(2-(dimethylamino)-1-(1H-imidazol-1-yl)propyl)naphthalen-2-yl)oxy)methyl)cyclohexanecarboxylate; methyl 1-(((6-(1-(1H-imidazol-1-yl)-2-(isopropyl(methyl)amino)propyl)naphthalen-2-yl)oxy)methyl)cyclopentanecarboxylate ; methyl 3-((6-(2-(diethylamino)-1-(1H-imidazol-1-yl)propyl)naphthalen-2-yl)oxy)-2,2-dimethylpropanoate; methyl 3-((6-(1-(1H-imidazol-1-yl)-2-(isopropyl(methyl)amino)propyl)naphthalen-2-yl)oxy)-2,2-dimethylpropanoate; methyl 3-((6-(2-(ethyl(methyl)amino)-1-(1H-imidazol-1-yl)propyl)naphthalen-2-yl)oxy)-2,2-dimethylpropanoate; methyl 3-((6-(2-(dimethylamino)-1-(1H-imidazol-1-yl)butyl)naphthalen-2-yl)oxy)-2,2-dimethylpropanoate; methyl 4-(((6-(2-(dimethylamino)-1-(1H-imidazol-1-yl)propyl)naphthalen-2-yl)oxy)methyl)benzoate; methyl 3-(((6-(2-(dimethylamino)-1-(1H-imidazol-1-yl)propyl)naphthalen-2-yl)oxy)methyl)benzoate; [6-(2-dimethylamino-1-imidazol-1-yl-propyl)-naphthalen-2-yloxy]-2,2-dimethyl-propionic acid; 2-[6-(2-dimethylamino-1-imidazol-1-yl-propyl)-naphthalen-2-yloxymethyl]-2-ethyl-butyric acid; 1-[6-(2-Dimethylamino-1-imidazol-1-yl-propyl)-naphthalen-2-yloxymethyl]-cyclopropanecarboxylic acid; [6-(2-dimethylamino-1-imidazol-1-yl-propyl)-naphthalen-2-yloxymethyl]-cyclobutanecarboxylic acid; [6-(2-Dimethylamino-1-imidazol-1-yl-propyl)-naphthalen-2-yloxymethyl]-cyclopentanecarboxylic acid; 1-[6-(2-dimethylamino-1-imidazol-1-yl-propyl)-naphthalen-2-yloxymethyl]-cyclohexanecarboxylic acid; 1-{6-[1-Imidazol-1-yl-2-(isopropylmethylamino)-propyl]-naphthalen-2-yloxymethyl}-cyclopentanecarboxylic acid; 3-[6-(2-diethylamino-1-imidazol-1-yl-propyl)-naphthalen-2-yloxy]-2,2-dimethyl-propionic acid; {6-[1-Imidazol-1-yl-2-(isopropylmethylamino)-propyl]-naphthalen-2-yloxy}-2,2-dimethyl-propionic acid; 3-6-[2-(Ethyl-methyl-amino)-1-imidazol-1-yl-propyl]-naphthalen-2-yloxy}-2,2-dimethyl-propionic acid; 3-[6-(2-Dimethylamino-1-imidazol-1-yl-propyl)-naphthalen-2-yloxy]-2,2-dimethyl-propionamide; 3-[6-(2-Dimethylamino-1-imidazol-1-yl-propyl)-naphthalen-2-yloxy]-2,2,N-trimethyl-propionamide ; 3-[6-(2-Dimethylamino-1-imidazol-1-yl-propyl)-naphthalen-2-yloxy]-2,2,N,N-tetramethyl-propionamide; 3-[6-(2-Dimethylamino-1-imidazol-1-yl-butyl)-naphthalen-2-yloxy]-2,2-dimethyl-propionic acid; 4-[6-(2-Dimethylamino-1-imidazol-1-yl-propyl)-naphthalen-2-yloxymethyl]-benzoic acid; 3-[6-(2-Dimethylamino-1-imidazol-1-yl-propyl)-naphthalen-2-yloxymethyl]-benzoic acid; 4-[6-(2-Dimethylamino-1-imidazol-1-yl-propyl)-naphthalen-2-yloxymethyl]-benzamide; 4-[6-(2-Dimethylamino-1-imidazol-1-yl-propyl)-naphthalen-2-yloxymethyl]-N-methyl-benzamide; 4-[6-(2-Dimethylamino-1-imidazol-1-yl-propyl)-naphthalen-2-yloxymethyl]-N,N-dimethyl-benzamide; or 1-[(6-Benzyloxy-naphthalen-2-yl)-(1-methyl-pyrrolidin-2-yl)-methyl]-1H-imidazole.

8. The method of claims 1-3 wherein the compound has a structure represented by formula (I-B): 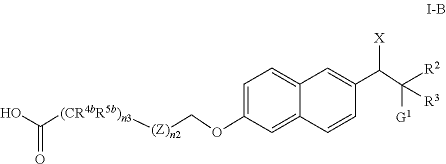
or an E or Z isomer thereof, syn or anti isomer thereof, an optically pure isomer thereof, or a pharmaceutically acceptable salt thereof, wherein:
X is substituted imidazolyl;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆ alkylamino carbonyl, diC₁₋₆ amino carbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸² (R⁹)_{n≡}, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
R^{4b} and R^{5b} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C1-10 alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC1-10 alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono (C₁₋₆alkyl)aminoC₁₋₆alkyl, di(C₁₋₆ alkyl)aminoC₁₋₆ alkyl, mono (aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or R^{4b} with R^{5b}, taken together with the respective carbon atom to which they are attached, form a carbonyl or 3-10 membered saturated or unsaturated ring, wherein said ring is optionally substituted with R⁶⁹;
R⁶⁷, R⁶⁸, and R⁶⁹ are each independently —OR⁷⁸, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R85, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄ alkyl) or —N(C₀₋₄alkyl)(C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl)(C₀₋₄alkyl) substituents; and
n2, n3, n4, n5, n6, and n7 are each independently equal to 0, 1 or 2.

9. The method of any one of claims 1-3 and 8 wherein the compound is [6-(2-dimethylamino-1-imidazol-1-yl-propyl)-naphthalen-2-yloxy]-2,2-dimethyl-propionic acid; 2-[6-(2-dimethylamino-1-imidazol-1-yl-propyl)-naphthalen-2-yloxymethyl]-2-ethyl-butyric acid; 1-[6-(2-dimethylamino-1-imidazol-1-yl-propyl)-naphthalen-2-yloxymethyl]-cyclopropanecarboxylic acid; [6-(2-dimethylamino-1-imidazol-1-yl-propyl)-naphthalen-2-yloxymethyl]-cyclobutanecarboxylic acid; [6-(2-Dimethylamino-1-imidazol-1-yl-propyl)-naphthalen-2-yloxymethyl]-cyclopentanecarboxylic acid; 1-[6-(2-dimethylamino-1-imidazol-1-yl-propyl)-naphthalen-2-yloxymethyl]-cyclohexanecarboxylic acid; 1-{6-[1-imidazol-1-yl-2-(isopropylmethylamino)-propyl]-naphthalen-2-yloxymethyl}-cyclopentanecarboxylic acid; 3-[6-(2-diethylamino-1-imidazol-1-yl-propyl)-naphthalen-2-yloxy]-2,2-dimethyl-propionic acid; {6-[1-imidazol-1-yl-2-(isopropylmethylamino)-propyl]-naphthalen-2-yloxy}-2,2-dimethyl-propionic acid; 3-6-[2-(ethyl-methyl-amino)-1-imidazol-1-yl-propyl]-naphthalen-2-yloxy}-2,2-dimethyl-propionic acid; 3-[6-(2-dimethylamino-1-imidazol-1-yl-butyl)-naphthalen-2-yloxy]-2,2-dimethyl-propionic acid; 4-[6-(2-dimethylamino-1-imidazol-1-yl-propyl)-naphthalen-2-yloxymethyl]-benzoic acid; or 3-[6-(2-dimethylamino-1-imidazol-1-yl-propyl)-naphthalen-2-yloxymethyl]-benzoic acid.

10. The method of any one of claims 1-3 wherein the compound has a structure represented by formula (II): 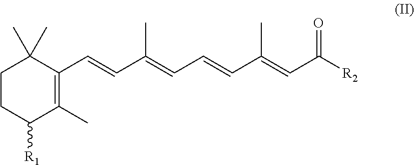
and E or Z isomers thereof, syn or anti isomers thereof, optically pure isomers thereof, or pharmaceutically acceptable salt thereof, and a pharmaceutically acceptable carrier therefor, wherein:
R¹ is an optionally substituted azole, sulfur, oxygen, nitrogen, pyridyl, acetylinic, cyclopropyl-amine, ester, oxime, cyano, amino, azido, cyclopropylamino, oxirane, aziridine, thiirane, thiol, alkylthiol, —OR₄ wherein R⁴ is hydrogen or an alkyl group, cyclopropylether, an oxygen containing group that forms, together with the 4-position carbon, an oxirane group; —NR⁵R⁶, where R⁵ and R⁶ are independently selected from the group consisting of hydrogen and alkyl, or R⁵ and R⁶ may together form a ring; and
R² is selected from the group consisting of hydroxyl, aminophenol, —OR³ and an azole group, wherein R³ is selected from the group consisting of alkyl, aryl and heterocyclic.

11. The method of claim 10 wherein the ring formed by R⁵ and R⁶ is an imidazolyl ring or a triazole ring.

12. The method of claim 110 wherein R² is a hydroxyl or —OCH₃.

13. The method of any one of claim 1-3 or 10 wherein the compound is selected from among (±)-4-(1H-imidazol-1-yl)methyl retinoate, (±)-4-(1H-imidazole-1-yl)retinoic acid, (±)-4-(1H-1,2,4-triazol-1-yl)methyl retinoate, (±)-4-(4H-1,2,4-triazole-4-yl)methyl retinoate, (±)-4-(1H-1,2,4-triazol-1-yl)retinoic acid, and (±)-4-(4H-1,2,4-triazol-4-yl)retinoic acid.

14. The method of any one of claims 1-13 wherein the chronic obstructive pulmonary disease is emphysema.

15. The method of any one of claims 1-14 wherein the chronic obstructive pulmonary disease is a result of tobacco abuse or smoking or exposure to second-hand smoke.

16. The method of any one of claims 1-13 wherein the disease or condition is fibrotic liver disease, hepatic ischemia-reperfusion injury, cerebral infarction, ischemic heart disease, renal disease or lung (pulmonary) fibrosis.

17. The method of any one of claims 1-13 wherein the disease or condition is liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs, transplants or grafts; ischemia/reperfusion injury; stroke; cerebrovascular disease; myocardial ischemia; atherosclerosis; renal failure; renal fibrosis or idiopathic pulmonary fibrosis.

18. The method of any one of claims 1-13 wherein the disease or condition is treatment of wounds for acceleration of healing; vascularization of a damaged and/or ischemic organ, transplant or graft; amelioration of ischemia/reperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; renal failure secondary to chronic diabetes and/or hypertension; amytrophic lateral sclerosis, muscular dystrophy, acute liver failure, acute liver injury, scleroderma, diabetes mellitus, multiple sclerosis, trauma to the central nervous system, and hereditary neurodegenerative disorders including the leukodystrophies such as metachromatic leukodystrophy, Refsum's disease, adrenoleukodystrophy, Krabbe's disease, phenylketonuria, Canavan disease, Pelizaeus-Merzbacher disease and Alexander's disease.
